(19) United States
(12) Patent Application Publication
(10) Pub. No.: US 2007/0043026 A1
(43) Pub. Date: Feb. 22, 2007

(54) Dopamine receptor modulators as antipsychotic agents

(76) Inventors: David Gwyn Cooper, Essex (GB); Ian Thomson Forbes, Essex (GB); Andrew Derrick Gribble, Essex (GB); Andrew Lightfoot, Essex (GB); Andrew H. Payne, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/513,919

(22) PCT Filed: May 9, 2003
(86) PCT No.: PCT/GB03/01983

§ 371 Date: May 23, 2005

(30) Foreign Application Priority Data
May 10, 2002 (GB) 0210762.1

(51) Int. Cl. A61K 31/55; A61K 31/47; C07D 223/16

(52) U.S. Cl. 514/217.01; 514/309; 514/416; 540/594

(57) ABSTRACT

The invention provides compounds of formula (I): 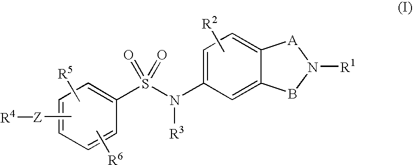 wherein

A and B represent the groups —(CH₂)ₘ—and —(CH₂)ₙ-respectively;

R¹ represents hydrogen or C₁₋₆alkyl;

R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸—(CH₂)ₚNR⁷COR⁸, optionally substituted aryl, optionally substituted heteroaryl or optionally substituted heterocyclyl;

R³ represents hydrogen or C₁₋₆alkyl;

R⁴ represents optionally substituted aryl or optionally substituted heteroaryl;

R⁵ and R⁶each independently represent hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or optionally substituted heterocyclyl;

R⁷ and R⁸ each independently represent hydrogen, C₁₋₆alkyl or, together with the nitrogen or other atoms to which they are attached, form an azacycloalkyl ring or an oxo-substituted azacycloalkyl ring; m and n independently represent an integer selected from 1 and 2; p independently represents an integer selected from 0, 1, 2 and 3; and either:
Z represents —CR⁹R¹⁰X— or —XCR⁹R¹⁰— and X represents oxygen, sulfur, —SO— or —SO₂, or
Z represents —CONR¹¹— or —NR⁹CO— and X represents —CH₂—, oxygen, sulfur, —SO— or —SO₂;

R⁹ and R¹⁰ each independently represent hydrogen, C₁₋₆alkyl or fluoro;

R¹¹ represents hydrogen or C₁₋₆alkyl; or a pharmaceutically acceptable salt or solvate thereof. The compounds of the invention are useful in therapy, in particular as antipsychotic agents.

This invention relates to novel compounds, pharmaceutical compositions containing them and their use in therapy, in particular as antipsychotic agents.

International patent application WO 02/74746 (Yamanouchi) discloses benzazepine derivatives that are 5-HT_{2C} receptor agonists and are said to be useful in the treatment of central nervous system disorders, especially in the treatment of sexual dysfunction.

International patent application WO 01/62737 (Ortho-McNeil) discloses amino pyrazole derivatives which are ligands for the neuropeptide Y subtype 5 receptor and are said to be useful in the treatment of disorders and disease associated with this receptor including, inter alia, obesity, anxiety, depression, pain and schizophrenia.

International patent application WO 01/85695 (Bristol-Myers Squibb) discloses tetrahydroisoquinoline analogues useful as growth hormone secretagogues. Such analogues are also said to be useful in the treatment of disorders including inter alia, obesity, schizophrenia, depression and Alzheimer's disease.

We have now found a novel group of phenylsulfonamide compounds which are useful particularly as antipsychotic agents.

According to the invention, there is provided a compound of formula (I):

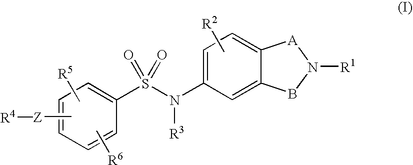

wherein

A and B represent the groups —(CH₂)ₘ— and —CH₂)ₘ— respectively;

R¹ represents hydrogen or C₁₋₆-alkyl;

R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or optionally substituted heterocyclyl;

R³ represents hydrogen or C₁₋₆alkyl;

R⁴ represents optionally substituted aryl or optionally substituted heteroaryl;

R⁵ and R⁶ each independently represent hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or optionally substituted heterocycyl;

R⁷ and R⁸ each independently represent hydrogen, C₁₋₆alkyl or, together with the nitrogen or other atoms to which they are attached, form an azacycloalkyl ring or an oxo-substituted azacycloalkyl ring;

m and n independently represent an integer selected from 1 and 2;

p independently represents an integer selected from 0, 1, 2 and 3; and either.
Z represents —CR⁹R¹⁰X— or —XCR⁹R¹⁰— and X represents oxygen, sulfur, —SO— or —SO₂, or
Z represents —CONR¹¹— or —NR¹¹CO—

R⁹ and R¹⁰ each independently represent hydrogen, C₁₋₆alkyl or fluoro;

R¹¹ represents hydrogen or C₁₋₆alkyl; or a pharmaceutically acceptable salt or solvate thereof.

It is to be understood that the present invention covers all combinations of particular and preferred groups described herein above.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy or hexyloxy.

As used herein, the term “C₁₋₆fluoroalkoxy” refers to a straight or branched alkoxy group containing the specified number of carbon atoms wherein any of the carbon atoms may be substituted by one or more fluorine atoms.

As used herein, the term “cycloalkyl” refers to a non-aromatic hydrocarbon ring containing the specified number of carbon atoms. For example, C₃₋₇cycloalkyl means a non-aromatic ring containing at least three, and at most seven, ring carbon atoms. Examples of “cycloalkyl” as used herein include, but are not limited to, cyclopropyl; cyclobutyl, cyclopentyl, cyclohexyl and cydoheptyl. A C₆₋₇cycloalkyl group is preferred.

As used herein, the term “halogen” refers to the elements fluorine, chlorine, bromine and iodine. Preferred halogens are fluorine, chlorine and bromine.

As used herein, the term “aryl” refers to a phenyl or a naphthyl ring.

As used herein, the term “heteroaryl” refers to a 5- or 6-membered heterocyclic aromatic ring or a fused bicyclic heteroaromatic ring system.

As used herein, the term “heterocyclyl” refers to a 3- to 7-membered monocyclic saturated ring containing at least one heteroatom independently selected from oxygen, nitrogen and sulfur. Examples of suitable heterocyclic rings include, but are not limited to, piperidine and morpholine.

As used herein, the term “5- or 6-membered heterocyclic aromatic ring” refers to a monocyclic unsaturated ring containing at least one heteroatom independently selected from oxygen, nitrogen and sulfur. Examples of suitable 5- and 6-membered heterocyclic aromatic rings include, but are not limited to, furyl, thienyl, pyrrolyl, oxazolyl, thiazolyl, imidazolyl, oxadiazolyl, thiadiazolyl, pyridyl, triazolyl, triazinyl, pyridazyl, pyrimidinyl, pyrazolyl, isothiazolyl and isoxazolyl.

As used herein, the term “fused bicyclic heteroaromatic ring system” refers to a ring system comprising one six-membered unsaturated ring and one 5- or 6-membered unsaturated ring fused together, the ring system containing at least one heteroatom independently selected from oxygen, nitrogen and sulfur. Examples of suitable fused bicyclic heteroaromatic ring systems include, but are not limited to, indolyl, benzofuranyl, quinolyl and benzothienyl.

As used herein, the term “azacycloalkyl ring” refers to a 4- to 7-membered monocyclic saturated ring containing one nitrogen atom. Examples of suitable azacycloalkyl rings are azetidine, pyrrolidine, piperidine and azepidine.

As used herein, the term “oxo-substituted azacycloalkyl ring” refers to an azacycloalkyl ring as defined above substituted by one oxo group. Examples of suitable oxo-substituted azacycloalkyl rings include, but are not limited to, azetidinone, pyrrolidinone, piperidinone and azepidinone.

As used herein, the term “substituted” refers to substitution with the named substituent or substituents, multiple degrees of substitution being allowed unless otherwise stated.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include water, methanol, ethanol and acetic acid. Most preferably the solvent used is water and the solvate may also be referred to as a hydrate.

It will be appreciated that for use in medicine the salts of formula (I) should be physiologically acceptable. Suitable physiologically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, malic, mandelic, acetic, fumaric, glutamic, lactic, citric, tartaric, benzoic, benzenesulfonic, p-toluenesulfonic, methanesulfonic or naphthalenesulfonic acid. Other non-physiologically acceptable salts e.g. oxalates, may be used, for example in the isolation of compounds of formula (l) and are included within the scope of this invention. Also included within the scope of the invention are solvates and hydrates of the compounds of formula (I).

Certain of the compounds of formula (I) may form acid addition salts with one or more equivalents of the acid. The present invention includes within its scope all possible stoichiometric and non-stoichiometric forms thereof.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

The groups R², R⁵, R⁶ and —Z—R⁴ may be located on any position on their respective phenyl rings.

When R², R⁵ and R⁶ represent optionally substituted aryl, optionally substituted heteroaryl or optionally substituted heterocyclyl, and R⁴ represents optionally substituted aryl or optionally substituted heteroaryl, the optional substituents may be selected from C₁₋₆alkyl, C₁₋₆alkoxy, halogen, trifluoromethyl, trifluoromethoxy, cyano and —S—C₁₋₆alkyl.

Preferably, R¹ represents hydrogen or C₁₋₄alkyl. More preferably, R¹ represents hydrogen, methyl, ethyl, n-propyl or isopropyl. Even more preferably, R¹ represents methyl.

Preferably, R² represents hydrogen, halogen, C₁₋₆alkyl, C₁₋₆alkoxy or diC₁₋₆alkylamino. More preferably, R² represents hydrogen, halogen, C₁₋₄alkyl, C₁₋₄alkoxy or diC₁₋₄alkylamino. Even more preferably, R² represents hydrogen, methyl, ethyl, iso-propyl, chloro, bromo, methoxy, ethoxy, isopropoxy or dimethylamino.

In a first embodiment of the invention, the R² group is located at the para-position relative to the group B i.e. a compound of formula (IA)

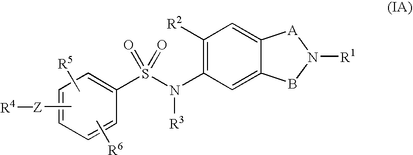

or a pharmaceutically acceptable salt or solvate thereof wherein groups A, B, Z and R¹ to R⁶ have any of the meanings as given hereinbefore.

In another embodiment of the invention, the R² group is located at the meta-position position relative to the group B i.e. a compound of formula (IB)

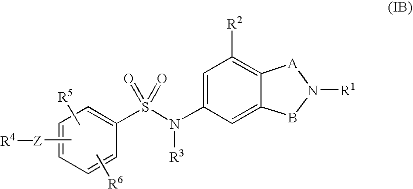

or a pharmaceutically acceptable salt or solvate thereof wherein groups A, B, Z and R¹ to R⁶ have any of the meanings as given hereinbefore.

When R² is located in the meta- or the para-position i.e. compounds of formula (IA) or (IB), R² is preferably hydrogen, methyl, ethyl, methoxy, chloro, bromo, ethoxy, isopropoxy or dimethylamino.

For compounds of the formula (I), (IA) or (IB), preferably, R³ represents hydrogen or C₁₋₄alkyl. More preferably, R³ represents hydrogen, methyl, ethyl, n-propyl or isopropyl. Even more preferably, R³ represents hydrogen.

For compounds of the formula (I), (IA) or (IB), preferably, R⁴ represents phenyl, thienyl or furyl, all of which may be optionally substituted. If R⁴ is optionally substituted, preferably R⁴ is mono- or di-substituted. More preferably, when R⁴ is phenyl, one of the optional substituents is located at the 4-position relative to the attachment of R⁴ to the rest of the molecule.

For compounds of the formula (I), (IA) or (IB), preferably, the optional substituents for the groups R², R⁴, R⁵ and R⁶ are selected from fluoro, chloro, bromo, methyl, ethyl, t-butyl, methoxy, trifluoromethyl, trifluoromethoxy, cyano, S-methyl and acetyl.

For compounds of the formula (I), (IA) or (IB), preferably, R⁵ and R⁶ independently represent hydrogen, methyl, fluoro or chloro.

For compounds of the formula (I), (IA) or (IB), preferably, R⁷ and R⁶ independently represent hydrogen or C₁₋₄alkyl. More preferably, R⁷ and R⁸ independently represent hydrogen or methyl.

For compounds of the formula (I), (IA) or (IB), preferably, Z represents —CR⁹R¹⁰X— or —X—CR⁹R¹⁰— wherein X represents oxygen, —NH or —NMe and R⁹ and R¹⁰ represent hydrogen or fluoro.

More preferably, Z represents —CR⁹R¹⁰X— wherein X represents oxygen and R⁹ and R¹⁰ both represent hydrogen i.e. —CH₂O—.

For compounds of the formula (I), (IA) or (IB), preferably, the —Z—R⁴ group is located either at the para-position in relation to the sulfonamide group i.e. a compound of formula (IC)

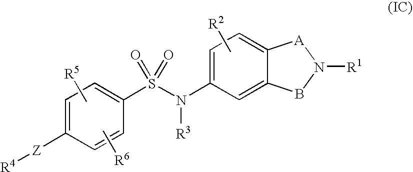

or at the meta-position in relation to the sulfonamide group i.e. a compound of formula (ID)

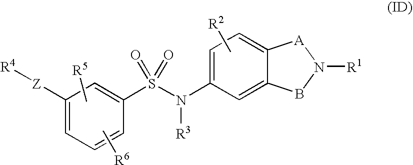

or a pharmaceutically acceptable salt or solvate thereof wherein groups A, B, Z and R¹ to R⁶ have any of the meanings as given hereinbefore.

For compounds of the formula (I), (IA), (IB), (IC) or (ID), preferably, p represents 0.

In another embodiment of the invention, m is 1 and n is 1 and the invention is a compound of formula (IE):

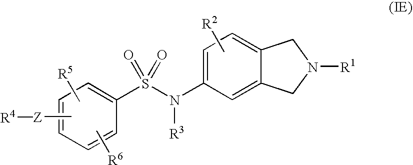

or a pharmaceutically acceptable salt or solvate thereof wherein groups Z and R¹ to R⁶ have any of the meanings as given hereinbefore.

In another embodiment of the invention, m is 2 and n is 1 and the invention is a compound of formula (IF):

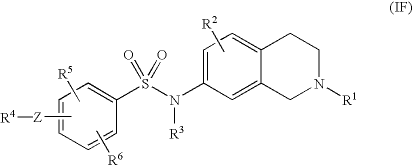

or a pharmaceutically acceptable salt or solvate thereof wherein groups Z and R¹ to R⁶ have any of the meanings as given hereinbefore.

In another embodiment of the invention, m is 1 and n is 2 and the invention is a compound of formula (IG):

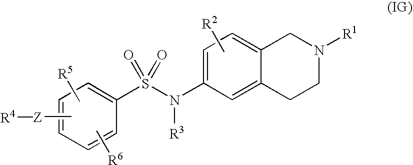

or a pharmaceutically acceptable salt or solvate thereof wherein groups Z and R¹ to R⁶ have any of the meanings as given hereinbefore.

In another embodiment of the invention, m is 2 and n is 2 and the invention is a compound of formula (IH):

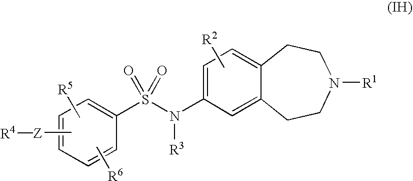

or a pharmaceutically acceptable salt or solvate thereof wherein groups Z and R¹ to R⁶ have any of the meanings as given hereinbefore.

In a preferred embodiment of the invention, compounds of the invention are of the formula (IH) or a pharmaceutically acceptable salt or solvate thereof wherein groups Z and R¹ to R⁶ have any of the meanings as given hereinbefore.

In a more preferred embodiment of the invention, compounds of the invention are of the formula (IH) and Z is —CH₂O— or a pharmaceutically acceptable salt or solvate thereof wherein R¹ to R⁶ have any of the meanings as given hereinbefore.

Particular compounds according to the invention include those incorporated in Tables 1 to 3 and those specifically exemplified and named hereinafter including, without limitation:—

4-benzyloxy-N-(8-bromo-2,3,4,5-tetrahydro-1H-benzo[d]azepin-7-yl)-benzenesulfonamide;

4-benzyloxy-N-(8-bromo-3-methyl-2,3,4,5-tetrahydro-1H-benzo[d]azepin-7-yl)-benzenesulfonamide hydrochloride;

4-(4-Chloro-phenoxymethyl)-N-(3-methyl-2,3,4,5-tetrahydro-1H-3-benzazepin-7-yl)-benzenesulfonamide hydrochloride;

4-(4-Fluoro-benzylamino)-N-(8-methoxy-3-methyl-2,3,4,5-tetrahydro-1H-3-benzazepin-7-yl)-benzenesulfonamide hydrochloride;

[(4-Fluoro-benzyl)-methyl-amino]-N-(8-methoxy-3-methyl-2,3,4,5-tetrahydro-1H-3-benzazepin-7-yl)-benzenesulfonamide hydrochloride;

4-[(4-Fluoro-phenylamino)-methyl]-N-(8-methoxy-3-methyl-2,3,4,5-tetrahydro-1H-3-benzazepin-7-yl)-benzenesulfonamide hydrochloride;

4-[(4-Fluoro-phenyl)-methyl-amino]-methyl}N-(8-methoxy-3-methyl-2,3,4,5-tetrahydro-1H-3-benzazepin-7-yl)-benzenesulfonamide hydrochloride;

4-{[(4-Fluoro-phenyl)-methyl-amino]-methyl}-N-(8-methoxy-3-methyl-2,3,4,5-tetrahydro-1H-3-benzazepin-7-yl)-benzenesulfonamide hydrochloride;

N-(8-Dimethylamino-3-methyl-2,3,4,5-tetrahydro-1H-3-benzazepin-7-yl)4-(4-fluoro-benzyloxy)-benzenesulfonamide hydrochloride;

N-(8-Dimethylamino-3-methyl-2,3,4,5-tetrahydro-1H-3-benzazepin-7-yl)-[(4-fluoro-benzyl)-methyl-amino]-benzenesulfonamide hydrochloride;

N-(8-Dimethylamino-3-methyl-2,3,4,5-tetrahydro-1H-3-benzazepin-7-yl)-[(4-fluoro-benzyl)-methyl-amino]-benzenesulfonamide hydrochloride; and

N-(Dimethylamino-methyl-2,3,4,5-tetrahydro-1H-3-benzazepin-7-yl)-4-{[(4-fluoro-phenyl)-methyl-amino]-methyl)benzenesulfonamide hydrochloride.

The compounds of the present invention may be in the form of their free base or physiologically acceptable salts thereof, particularly the monohydrochloride or monomesylate salts.

The present invention also provides a general process (A) for preparing compounds of formula (I) which process comprises: reacting a compound of formula (II)

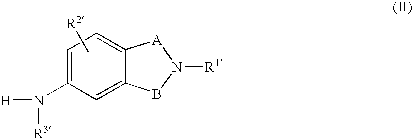

with a compound of formula (Ill)

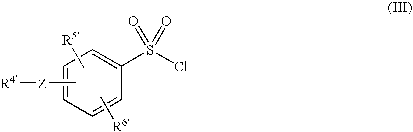

wherein R^{1′}-R^{6′} represent R¹ to R⁶ as hereinbefore defined or are groups that may be readily convertible to R¹ to R⁶.

This general method (A) can be conveniently performed by mixing the two components in a suitable solvent such as pyridine or dichloromethane (in the presence of a base), at 0° C.

The present invention also provides a general process (B) for preparing compounds of formula (I), which process comprises:

reacting a compound of formula (IV)

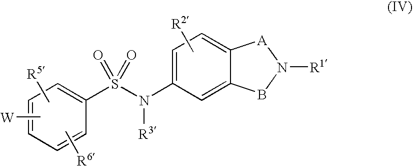

with a compound of formula (V)
R^{4′}—V (V)

wherein A, B and Z are as hereinbefore defined and R^{1′}-R^{6′} represent R¹ to R⁶ as hereinbefore defined or are groups that may be readily convertible to R¹ to R⁶, and V and W contain the appropriate functionalities to generate the Z linker. For example,

a) W═OH and V═CH₂Br using standard alkylation conditions such as sodium hydride or potassium carbonate in dimethylformamide. Protection of R³═H may be necessary.

b) W═CH₂Br and V═OH using standard alkylation conditions such as sodium hydride or potassium carbonate in dimethylformamide. Protection of R³═H may be necessary

c) W═OH and V═CH₂OH using standard Mitsunobu conditions i.e. treatment with diisopropyl azodicarboxylate/triphenylphosphine in tetrahydrofuran at room temperature. Protection of R³═H may be necessary.

d) W═CH₂OH and V═OH using standard Mitsunobu conditions i.e. treatment with diisopropyl azodicarboxylate/triphenylphosphine in tetrahydrofuran at room temperature. Protection of R³═H may be necessary.

e) W═COCH and V═Br using standard Sonagashira conditions, followed by catalytic hydrogenation of the triple bond.

f) W═NH₂ and V═CO₂H, or V═NH₂ and W═CO₂H using standard amide forming conditions, e.g. Di-isopropylcarbodiimide and HOBT in dichloromethane at room temperature.

g) W═F and V═CH₂SH using standard aromatic nucleophilic substitution conditions i.e. NaH in dimethylformamide at room temperature, followed by oxidation of the sulfide.

The present invention also provides a general process (C) for preparing compounds of formula (I) which process comprises:

converting a compound of formula (I)

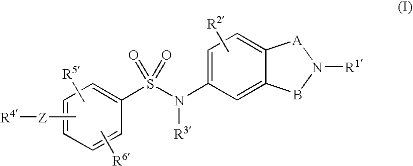

into another compound of formula (I) wherein A, B and Z are as hereinbefore defined and R^{1′}-R^{6′} represent R¹ to R⁶ as hereinbefore defined or are groups that may be readily convertible to R¹ to R⁶, by substituting the group R¹ or the group R³ using conventional techniques.

Interconversion of one of the R^{1′} to R^{5′} groups to the corresponding R¹ to R⁵ groups typically arises when one compound of formula (I) is used as the immediate precursor of another compound of formula (I), or when it is easier to introduce a more complex or reactive substituent at the end of a synthetic sequence.

For example, conversion of R^{1′} from a t-butoxycarbonyl (BOC) group to hydrogen is conducted by the treatment of the N—BOC protected compound with hydrogen chloride in ethanol or dioxan at room temperature.

Conversion of R^{1′} from hydrogen to an alkyl group is conducted by the treatment of the NH compound with the appropriate aldehyde in dichloroethane in the presence of a reducing agent, such as sodium triacetoxyborohydride, or by the treatment of the NH compound with the appropriate alkyl halide, such as iodomethane, under standard alkylation conditions (potassium carbonate in DMF at 60° C.).

Conversion of R^{3′} from hydrogen to an alkyl group is conducted by the treatment of the sulfonamide NH compound with the appropriate alcohol, such as methanol, under Mitsunobu conditions i.e. treatment with diisopropyl azodicarboxylate/triphenylphosphine and methanol in tetrahydrofuran at room temperature.

Compounds of formula (II) are known in the literature or may be prepared by known processes, for example, reduction of the corresponding nitro compound as disclosed in WO 99/14197, or by procedures analogous to these procedures. Suitable examples of an R^{1′} protecting group are trifluoroacetyl or the t-butoxycarbonyl (BOC) group.

Compounds of formula (III) are commercially available or may be prepared by established procedures, for example chlorosulfonylation of a suitable substituted aromatic precursor, using chlorosulfonic acid, for example as described in U.S. Pat. No. 5,872,138.

Alternatively, compounds of formula (III) wherein Z represents CR⁹R¹⁰, R^{4′}-R^{6′} represent R⁴ to R⁶ as hereinbefore defined or are groups that may be readily convertible to R⁴ to R⁶ and R⁹ and R¹⁰ are as hereinbeforehand described, may be prepared from ortho, meta or para mercaptophenol (VI) by a novel 3 step process.

Therefore, the present invention also provides a general process (D) for preparing compounds of formula (III) which process comprises:

(a) oxidative dimerisation of a compound of formula (VI)

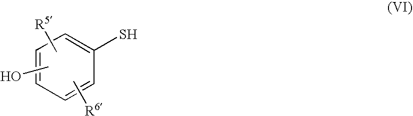

with, for example, iodine in methanol;

(b) alkylating the resulting symmetrical disulfide (VII) 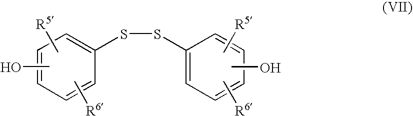 on oxygen using base, for example sodium hydride and an alkylating agent for example, a substituted benzyl bromide; and

(c) oxidative cleavage of the resulting compound of formula (VIII)

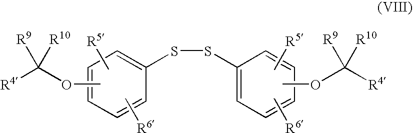

using, for example N-chlorosuccinimide.

Compounds of formula (IV) may be prepared from compounds of formula (II) by the treatment with the appropriate 4-substituted benzenesulfonyl chloride using standard conditions, for example in pyridine or dichloromethane in the presence of a base such as triethylamine at room temperature.

Compounds of formula (V) are in most cases commercially available or may be prepared by known methodology.

Compounds of formula (I) have been found to exhibit affinity for dopamine receptors, in particular the D₃ and D₂ receptors, and are useful in the treatment of disease states which require modulation of such receptors, such as psychotic conditions. Many of the compounds of formula (I) have also been found to have greater affinity for dopamine D₃ than for D₂ receptors. The therapeutic effect of currently available antipsychotic agents (neuroleptics) is generally believed to be exerted via blockade of D₂ receptors; however this mechanism is also thought to be responsible for undesirable extrapyramidal side effects (eps) associated with many neuroleptic agents. Without wishing to be bound by theory, it has been suggested that blockade of the dopamine D₃ receptor may give rise to beneficial antipsychotic activity without significant eps. (see for example Sokoloff et al, Nature, 1990; 347: 146-151; and Schwartz et al, Clinical Neuropharmacology, Vol 16, No. 4, 295-314, 1993). Additionally, certain compounds of formula (I) have antagonist affinity for the serotonin 5-HT_{2C}, 5-HT_{2A} and 5-HT₆ receptors. These additional properties may give rise to enhanced anti-psychotic activity (e.g. improved effects on cognitive dysfunction) and/or reduced eps. These could include, but are not limited to, attenuation of cognitive symptoms via 5-HT₆ receptor blockade (see Reavill, C. and Rogers, D. C., 2001, Investigational Drugs 2, 104-109), and reduced anxiety (see for example Kennett et al., Neuropharmacology April-May 1997; 36 (4-5): 609-20), protection against EPS (Reavill et al., Brit. J. Pharmacol., 1999; 126: 572-574) and antidepressant activity (Bristow et al., Neuropharmacology 39:2000; 1222-1236) via 5-HT_{2C} receptor blockade.

Compounds of formula (I) may also exhibit affinity for other receptors not mentioned above, resulting in beneficial antipyschotic activity.

The compounds of formula (I) are of use as antipsychotic agents for example in the treatment of schizophrenia, schizo-affective disorders, schizophreniform diseases, psychotic depression, mania, acute mania, paranoid and delusional disorders. Furthermore, they may have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D₃ receptors, it could also be envisaged that the compounds could also have utility for the treatment of substance abuse where it has been suggested that D3 receptors are involved (e.g. see Levant, 1997, Pharmacol. Rev., 49, 231-252). Examples of such substance abuse include alcohol, cocaine, heroin and nicotine abuse. Other conditions which may be treated by the compounds include dyskinetic disorders such as Parkinson's disease, neuroleptic-induced parkinsonism and tardive dyskinesias; depression; anxiety; agitation; tension; social or emotional withdrawal in psychotic patients; cognitive impairment including memory disorders such as Alzheimer's disease; psychotic states associated with neurodegeneraltive disorders, e.g. Alzheimer's disease; eating disorders; obesity; sexual dysfunction; sleep disorders; emesis; movement disorders; obsessive-compulsive disorders; amnesia; aggression; autism; vertigo; dementia; circadian rhythm disorders; and gastric motility disorders e.g. IBS.

Therefore, the invention provides a compound of formula (I) as hereinbefore described or a pharmaceutically acceptable salt or solvate thereof for use in therapy.

The invention also provides a compound of formula (I) or a pharmaceutically acceptable salt or solvate thereof for use in the treatment of a condition which requires modulation of a dopamine receptor.

The invention also provides a compound of formula (I) as hereinbefore described or a pharmaceutically acceptable salt or solvate thereof for use in the treatment of psychotic disorders, schizophrenia, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides the use of a compound of formula (I) as hereinbefore described or a pharmaceutically acceptable salt or solvate thereof in the manufacture of a medicament for the treatment of a condition which requires modulation of a dopamine receptor.

The invention also provides the use of a compound of formula (I) as hereinbefore described or a pharmaceutically acceptable salt or solvate thereof in the manufacture of a medicament for the treatment of psychotic disorders, schizophrenia, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides a method of treating a condition which requires modulation of a dopamine receptor, which comprises administering to a mammal in need thereof an effective amount of a compound of formula (I) as hereinbefore described or a pharmaceutically acceptable salt or solvate thereof.

In a further aspect, the invention provides a method of treating psychotic disorders, schizophrenia, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

A preferred use for dopamine antagonists according to the present invention is in the treatment of psychotic disorders, schizophrenia, Parkinsons disease, substance abuse, dyskinetic disorders, depression, bipolar disorder, anxiety and cognitive impairment.

“Treatment” includes prophylaxis, where this is appropriate for the relevant condition(s).

It will be appreciated by those skilled in the art that the compounds according to the invention may advantageously be used in conjunction with one or more other therapeutic agents, for instance, different antidepressant agents such as 5HT₃ antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H₃ antagonists, 5HT_{1A} antagonists, 5HT_{1B} antagonists, 5HT_{1D} antagonists, D₁ agonists, M₁ agonists and/or anticonvulsant agents.

Suitable 5HT₃ antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline. Suitable dopaminergic antidepressants which may be used in combination with a compound of the invention include bupropion and amineptine.

Suitable anticonvulsant agents which may be used in combination of the compounds of the inventions include for example divalproex, carbamazepine and diazepam.

It will be appreciated that the compounds of the combination or composition may be administered simultaneously (either in the same or different pharmaceutical formulations), separately or sequentially.

For use in medicine, the compounds of the present invention are usually administered as a standard pharmaceutical composition. The present invention therefore provides in a further aspect a pharmaceutical composition comprising a compound of formula (I) as hereinbefore described or a pharmaceutically (i.e. physiologically) acceptable salt thereof and a pharmaceutically (i.e. physiologically) acceptable carrier. The pharmaceutical composition can be for use in the treatment of any of the conditions described herein.

The compounds of formula (I) may be administered by any convenient method, for example by oral, parenteral (e.g. intravenous), buccal, sublingual, nasal, rectal or transdermal administration and the pharmaceutical compositions adapted accordingly. The compounds of formula (I) as hereinbefore described and their pharmaceutically acceptable salts which are active when given orally can be formulated as liquids or solids, for example syrups, suspensions or emulsions, tablets, capsules and lozenges.

A liquid formulation will generally consist of a suspension or solution of the compound or pharmaceutically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; altematively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Altematively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochloro-hydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. Preferably the composition is in unit dose form such as a tablet, capsule or ampoule.

Each dosage unit for oral administration contains preferably from 1 to 250 mg (and for parenteral administration contains preferably from 0.1 to 25 mg) of a compound of the formula (I) or a pharmaceutically acceptable salt thereof calculated as the free base.

The pharmaceutically acceptable compounds of the invention will normally be administered in a daily dosage regimen (for an adult patient) of, for example, an oral dose of between 1 mg and 500 mg, preferably between 10 mg and 400 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a pharmaceutically acceptable salt thereof calculated as the free base, the compound being administered 1 to 4 times per day. Suitably the compounds will be administered for a period of continuous therapy, for example for a week or more.

No toxicological effects are indicated/expected when a compound of the invention is administered in the above mentioned dosage range.

Biological Test Methods

Binding Experiments on Cloned Dopamine (e.g. D2 and D3) Receptors

The ability of the compounds to bind selectively to human D2/D3 dopamine receptors can be demonstrated by measuring their binding to cloned receptors. The inhibition constants (Kᵢ) of test compounds for displacement of [¹²⁵I]-Iodosulpride binding to human D2/D3 receptors expressed in CHO cells were determined as follows. The cell lines were shown to be free from bacterial, fungal and mycoplasmal contaminants, and stocks of each were stored frozen in liquid nitrogen. Cultures were grown as monolayers or in suspension in standard cell culture media. Cells were recovered by scraping (from monolayers) or by centrifugation (from suspension cultures), and were washed two or three times by suspension in phosphate buffered saline followed by collection by centrifugation. Cell pellets were stored frozen at −80° C. Crude cell membranes were prepared by homogenisation followed by high-speed centrifugation, and characterisation of cloned receptors achieved by radioligand binding.

Preparation of CHO cell membranes: Cell pellets were gently thawed at room temperature, and resuspended in about 20 volumes of ice-cold Extraction buffer; 5 mM EDTA, 50 mM Trizma pre-set crystals (pH7.4 @37° C.), 1 mM MgCl₂, 5 mM KCl and 120 mM NaCl. The suspension was homogenised using an Ultra-Turrax at full speed for 15 seconds. The homogenate was centrifuged at 18,000 r.p.m. for 15 min at 4° C. in a Sorvall RC5C centrifuge. Supernatant was discarded, and homogenate re-suspended in extraction buffer then centrifugation was repeated. The final pellet was resuspended in 50 mM Trizma pre-set crystals (pH 7.4@37° C.) and stored in 1 ml aliquot tubes at −80° C. (D2=3.0E+08 cells, D3=7.0E+07 cells and D4=1.0E+08 cells). The protein content was determined using a BCA protocol and bovine serum albumin as a standard (Smith, P. K., et al., Measurement of protein using bicinchoninic acid. Anal. Biochem. 150, 76-85 (1985)).

Binding experiments: Crude D2/D3 cell membranes were incubated with 0.03 nM [¹²⁵I]-Iodosulpride (˜2000 Ci/mmol; Amersham, U. K., and the test compound in a buffer containing 50 mM Trizma pre-set crystals (pH 7.4@37° C.), 120 mM NaCl, 5 mM KCl, 2 mM CaCl₂, 1 mM MgCl₂, 0.3% (w/v) bovine serum albumin. The total volume is 0.2 ml and incubated in a water bath at 37° C. for 40 minutes. Following incubation, samples were filtered onto GF/B Unifilters using a Canberra Packard Filtermate, and washed four times with ice-cold 50 mM Trizma pre-set crystals (pH 7.4@37° C.). The radioactivity on the filters was measured using a Canberra Packard Topcount Scintillation counter. Non-specific binding was defined with 10 μM SKF-102161 (YM-09151). For competition curves, 10 serial log concentrations of competing cold drug were used (Dilution range: 10 μM-10 μM). Competition curves were analysed using Inflexion, an iterative curve fitting programme in Excel. Results were expressed as pKᵢ values where pKᵢ=−log10[Ki].

The exemplified compounds have pKᵢ values within the range of 6.0-9.2 at the dopamine D₃ receptor.

The exemplified compounds have pKᵢ values within the range of 5.6-8.0 at the dopamine D₂ receptor.

Binding Experiments on Cloned 5-HT₆ Receptors

Compounds can be tested following the procedures outlined in WO 98/27081.

The exemplified compounds have pKᵢ values within the range of 6.9-9.4 at the serotonin 5-HT₆ receptor.

Binding Experiments on Cloned 5-HT_{2C} and 5-HT_{2A} Receptors

Compounds can be tested following the procedures outlined in WO 94/04533.

The exemplified compounds have pKᵢ values within the range of 7.1-8.3 at the serotonin 5-HT_{2C} and 5-HT_{2A} receptor.

The invention is further illustrated by the following non-limiting examples:

Description 1

### 7-Amino-1,2,4,5-tetrahydro-3-benzazepine-3-carboxylic acid tert-butyl ester (D1)

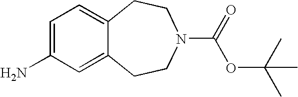

The title compound was prepared using a similar methodology to that described in EP 284384. MH⁺ 263

Description 2

### 7-Amino-8-bromo-1,2,4,5-tetrahydro-3-benzazepine-3-carboxylic acid tert-butyl ester (D2)

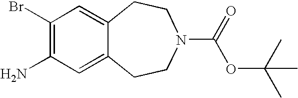

The aniline D1 (5 g, 19 mmol) was dissolved in dry CH₃CN (100 ml) and the solution was cooled to −15° C. A solution of N-bromosuccinimide (1.03 eq, 19.6 mmol, 3.48 g, in 70 ml of dry CH₃CN) was added dropwise at −15° C. to the solution containing the aniline, over 20 min. After the addition, the reaction mixture was left to warm up to room temperature for 10 min and then it was poured onto water/brine (150 ml+15 ml). The aqueous was extracted with EtOAc (100 ml, 50 ml), the organics were combined, dried over Na₂SO₄, filtered and the solvent was evaporated to afford the crude product. Chromatography on silica eluting with 5-30% EtOAc/n-hexane afforded the title compound (1.3 g). (M⁺−Boc)=241.

Description 3

### 7-Amino-8-chloro-1,2,4,5-tetrahydro-3-benzazepine-3-carboxylic acid tert-butyl ester (D3)

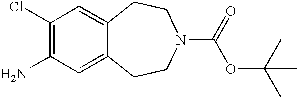

To a stirred solution of D1 (10 g, 38 mmol) in acetonitrile (300 ml) at 0° C. was added N-chlorosuccinimide (6.6 g, 49 mmol) portionwise over 10 minutes. The resulting solution was stirred overnight at room temperature then water (500 ml) and EtOAc (500 ml) were added. The organic layer was separated, dried over magnesium sulfate and concentrated in vacuo to give a dark brown oil. This was purified by column chromatography using 20% diethyl ether/hexane as the eluant to give the title compound as an orange glassy solid. (MH-Boc)⁺ 197.1, 199.1

Description 4

### 7-Amino-8-ethyl-1,2,4,5-tetrahydro-3-benzazepine-3-carboxylic acid tert-butyl ester (D4)

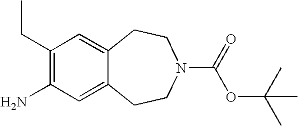

### a) 7-Hydroxy-1,2,4,5-tetrahydro-benzo[d]azepine-3-carboxylic acid tert-butyl ester

The title compound was prepared according to the procedure in WO 00/21951.

### b) 7-Hydroxy-8-nitro-1,2,4,5-tetrahydro[d]azepine-3-carboxylic acid tert-butyl ester

Nitration of D4a was carried out by adding 70% aqueous nitric acid (8 g) dissolved in glacial acetic acid (100 mL)/acetic anhydride (10 mL) to the phenol D4a (20 g) dissolved in AcOH (200 mL)/acetic anhydride (20 mL) at 0° C. Aqueous work-up followed by chromatography on silica gel using 0-20% EtOAc/n-hexane as eluant afforded the title compound (11 g).

### c) 7-Nitro-8-trifluoromethanesulfonyloxy-1,2,4,5-tetrahydro[d]azepine-3-carboxylic acid tert-butyl ester

D4b (8.4 g) was dissolved in acetone (300 mL) and cooled to 0° C. Trifluoromethanesulfonyl chloride (4.4 ml) was added and the resultant mixture stirred at room temperature for 2 h. Evaporation in vacuo followed by basic aqueous work-up afforded the title compound (12 g).

### d) 7-nitro-8-vinyl-1,2,4,5-tetrahydro[d]azepine-3-carboxylic acid tert-butyl ester

A mixture of D4c (500 mg ), vinyl tri-n-butyltin (0.4 mL), lithium chloride (145 mg), palladium tetrakistriphenylphosphine (131 mg) and 2,6-di-tert-butylphenol (4 mg ) in 1,4-dioxan (4 mL) was heated at 160° C. for 0.5 h in a sealed tube in a Smith microwave reactor. Aqueous work-up followed by chromatography using 0-20% EtOAcdn-hexane as eluant gave the title compound (260 mg).

### e) 7-amino-8-ethyl-1,2,4,5-tetrahydro[d]azepine-3-carboxylic acid tert-butyl ester

Hydrogenation of D4d (260 mg) at 50 psi in ethanol (40 mL) over 10% palladium on charcoal (100 mg, paste) at room temperature afforded the title compound (190 mg).

Description 5

### 7-Amino-8-methoxy-1,2,4,5-tetrahydro-3-benzazepine-3-carboxylic acid tert-butyl ester (D5)

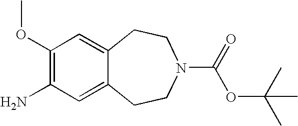

### a) 7-Hydroxy-1,2,4,5-tetrahydro-benzo[d]azepine-3-carboxylic acid tert-butyl ester

The title compound was prepared according to the procedure in WO 00/21951

### b) 7-Methoxy-1,2,4,5-tetrahydro-benzo[d]azepine-3-carboxylic acid tert-butyl ester

Reaction of the phenol D5a with potassium carbonate/methyl iodide in dimethylformamide afforded the title compound. MH⁺ 278.

### c) 7-Methoxy-8-nitro-1,2,4,5-tetrahydro-benzo[d]azepine-3-carboxylic acid tert-butyl ester

Nitration of D5b was carried out using a solution of nitric acid and acetic anhydride; the crude product was purified by chromatography on silica gel using EtOAc/n-hexane as eluant to afford the title compound. M⁺−C(CH₃)₃+2H=267

### d) 7-Amino-8-methoxy-1,2,4,5-tetrahydro-3-benzazepine-3-carboxylic acid tert-butyl ester

Hydrogenation of D5c at 50 psi in ethanol over 10% palladium on charcoal at room temperature afforded the title compound. MH⁺ 293.

Description 6

### 7-Amino-8-methyl-1,2,4,5-tetrahydro-3-benzazepine-3-carboxylic acid tert-butyl ester (D6)

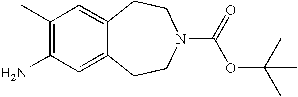

### a) 7-Methyl-8-nitro-1,2,4,5-tetrahydro[d]azerine-3-carboxylic acid tert-butyl ester

A mixture of D4c (1.0 g), tetramethyltin (0.6 mL), lithium chloride (0.29 g), palladium tetrakistriphenylphosphine (0.13 g) and 2,6-di-tert-butylphenol (cat. ) in 1,4-dioxan (4 mL) was heated at 160° C. for 0.5 h in a sealed tube in a Smith microwave reactor. Aqueous work-up followed by chromatography using 0-20% EtOAc/n-hexane as eluant gave the title compound (0.44 g).

### b) 7-amino-8-ethyl-1,2,4,5-tetrahydro[d]azepine-3-carboxylic acid tert-butyl ester

Hydrogenation of D6a (440 mg) at 50 psi in ethanol (100 mL) over 10% palladium on charcoal (200 mg, paste) at room temperature afforded the title compound (330 mg).

Description 7

### 9-Chloro-3-methyl-2,3,4,5-tetrahydro-1H-benzo[d]azepin-7-ylamine (D7)

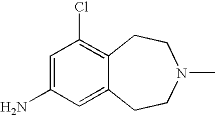

### a) 3-Acetyl-7-nitro-1,2,4,5-tetrahydro-3-benzazepine

The title compound was prepared according to a similar procedure described in J. Heterocycl. Chem. 1971, 8(5), 779.

### b) 3-Acetyl-7-nitro-9-iodo-1,2,4,5-tetrahydro-3-benzazepine

D7a (22.4 g) in trifluoromethanesulphonic acid (150 ml) was treated with N-iodosuccinimide (40 g) portionwise over 5 days. Aqueous workup gave the crude title compound (25 9). MH⁺ 361.

### c) 7-nitro-9-iodo-1,2,4,5-tetrahydro-3-benzazepine

Crude D7b (25 g) was heated to 120° C. in concentrated hydrochloric acid (1 L) for 12 h. Basic aqueous workup followed by chromatography using 5% methanol/dichloromethane as eluent gave the title compound (7 g). MH⁺ 319.

### d) 3-Methyl-7-nitro-9-iodo-1,2,4,5-tetrahydro-3-benzazepine

D7c (7.3 g) was treated with formalin (37% aqueous, 20 ml) in dichloroethane (30 ml) for 0.5 h, followed by sodium triacetoxyborohydride (7 g). Chromatography using 1% methanol/dichloromethane as eluent and recrystallisation from dichloromethane/hexane gave the title compound (1.9 g). MH⁺ 333.

### e) 3-Methyl-7-nitro-9-chloro-1,2,4,5-tetrahydro-3-benzazepine

Reaction of D7d (0.8 g) with copper(l) chloride (1.68 g) in dimethylformamide (15 ml) at 120° C. for 2 h followed by chromatography using 1-3% methanol/dichloromethane as eluent gave the title compound (0.3 g). MH⁺ 241.

### f) 3-Methyl-7-amino-9-chloro-1,2,4,5-tetrahydro-3-benzazepine

Hydrogenation of D7e (0.3 g) at 1 atmosphere in ethanol over 10% rhodium on charcoal at room temperature afforded the title compound (0.19 g). MH⁺ 211.

Description 8

### 9-Bromo-3-methyl-2,3,4,5-tetrahydro-1H-benzo[d]azepin-7-ylamine (D8)

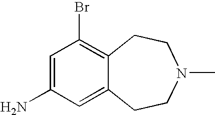

### a) 3-Methyl-7-nitro-9-iodo-1,2,4,5-tetrahydro-3-benzazepine

The title compound was prepared according to the procedure described in D7d.

### b) 3-Methyl-7-nitro-9-bromo-1,2,4,5-tetrahydro-3-benzazepine

Reaction of D8a (1 g) with copper(l) bromide (3 g) in dimethylformamide (10 ml) at reflux for 3 h followed by chromatography using 1-3% methanol/dichloromethane as eluent gave the title compound (0.23 g). MH⁺ 286.

### c) 3Methyl-7-amino-9-bromo-1,2,4,5-tetrahydro-3-benzazepine

Reduction of the nitro group was achieved by treating D8b (0.23 g) in ethanol (6 ml), water (3 ml) and acetic acid (0.5 ml) with iron powder (180 mg) at reflux for 1 h. Basic aqueous workup and filtering gave the title compound (0.19 g). MH⁺ 256.

Description 9

### 7-Amino-3,4-dihydro-1H-isoquinoline-2-carboxylic acid tert-butyl ester (D9)

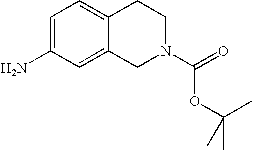

The title compound was prepared using a similar methodology to that described in WO 9914197. MH⁺ 249

Description 10

### 4-Benzyloxy-benzenesulfonyl chloride (D10)

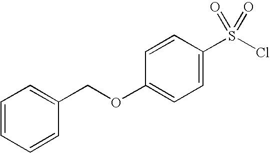

The title compound was prepared from benzyl phenyl ether and sulfuryl chloride according to the procedure in U.S. Pat. No. 5,872,138

Description 11

### 4-(4-Chloro-benzyloxy)-benzenesulfonyl chloride (D11)

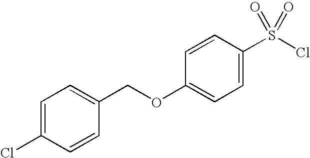

The title compound was prepared from 4-chlorobenzyl phenyl ether and sulfuryl chloride using a procedure similar to that for D9.

Description 12

### 4-(4-Fluoro-benzyloxy)-benzenesulfonyl chloride (D12)

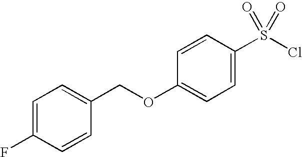

A stirred solution of bis-(4-hydroxyphenyl)disulfide (1.33 g), in dimethylformamide (50 mL) was treated with sodium hydride (60% in oil) (0.46 g) over 20 minutes. The solution was then treated with 4-fluorobenzyl bromide (1.6 mL) and stirred for 1 hour. The solution was poured into water and extracted with ether. The ether extract was then washed with brine and solvent evaporation gave bis-[4-(4-fluorobenzyloxy)-phenylldisulfide as a white solid from hexane (1.97 g)

A stirred solution of bis-[4-(4-fluorobenzyloxy)-phenyl]disulfide (0.466 g), acetic acid (20 mL) and water (5 mL) was cooled in a ice bath and treated with N-chlorosuccinimide (0.655 g). The solution was stirred for 1 hour, poured into water, and extracted with ethyl acetate. The extracts were then washed with brine. Solvent evaporation gave the title compound as a white solid from hexane (0.43 g)

Description 13

### 3-Benzyloxy-benzenesulfonyl chloride (D13)

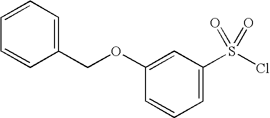

The title compound was prepared according to the procedure in Bioorganic Med Chem Lett 1995, 5(4), 319.

Descriptions 14 (a-p)

The following substituted benzenesulfonyl chlorides were prepared using a method similar to Description 12

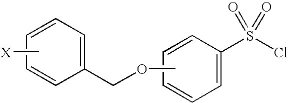

| | ortho/meta/para | |
|---|---|---|
| Description 14 | to SO₂Cl group | X |
| a | para | 3-Cl |
| b | para | 2-Me |
| c | para | 3-Me |
| d | para | 3,4-diF |
| e | para | 2,4-diF |
| f | para | 2-F |
| g | para | 3-F |
| h | para | 4-CF₃ |
| i | para | 4-Me |
| j | para | 4-Br |
| k | meta | 4-F |
| l | meta | 4-Cl |
| m | meta | H |
| n | ortho | 4-F |
| o | ortho | 4-Cl |
| p | ortho | H |

Description 15

### 7-Amino-8-isopropoxy-1,2,4,5-tetrahydro-3-benzazepine-3-carboxylic acid tert-butyl ester (D15)

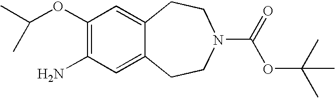

The title compound was prepared in accordance with Description 5, but methyl iodide was replaced with isopropyl iodide for the alkylation of the phenol. ¹H NMR (CDCl₃) δ 6.57 (1H, s), 6.50 (1H, s), 4.46 (1H, hept, J=6.1 Hz), 3.68 (2H, s), 3.51 (4H, m), 2.74 (4H, m), 1.48 (9H, s), 1.33 (6H, d, J=6.1 Hz).

Description 16

### 7-Amino-8-dimethylamino-1,2,4,5-tetrahydro-benzo[d]azepine-3-carboxylic acid tert-butyl ester (D16)

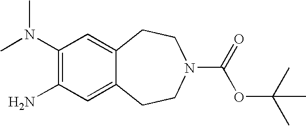

### a) 7-Hydroxy-1,2,4,5-tetrahydro-benzo[d]azepine-3-carboxylic acid tert-butyl ester (D16a)

The title compound was prepared according to the procedure described in WO 00/21951 i.e. 7-Methoxy-2,3,4,5-tetrahydro-1H-3-benzazepine (10 g) in 48% aqueous hydrobromic acid (350 ml) was allowed to stir at 100° C. for 4 h. The mixture was allowed to cool to 20° C. then evaporated to dryness, giving the crude hydroxy compound as a brown solid (14.5 g). This solid was dissolved in tetrahydrofuran (100 ml) and water (70 ml) and triethylamine (8 g) was added dropwise, followed by a solution of di-tert-butyl dicarbonate (14 g) in tetrahydrofuran (20 ml). The resulting mixture was allowed to stir at 20° C. for 16 h then partitioned between ethyl acetate (200 ml) and water (200 ml). The aqueous layer was extracted with ethyl acetate (100 ml). The combined organic extracts were washed with saturated aqueous sodium bicarbonate (100 ml), dried over anhydrous sodium sulfate and evaporated to dryness. The resulting oil was purified by chromatography over silica gel, eluting with 10-30% ethyl acetate in hexane, affording the title compound D15a as a white solid (8 g), MS (API⁺): Found 164 (MH⁺-Boc). C₁₅H₂₁NO₃ requires 263. ¹H NMR: δ CDCl₃ 1.48 (9H, s), 2.75-2.87 (4H, m), 3.40-3.60 (4H, m), 4.95 (1H, s), 6.50-6.62 (2H, m), 6.96 (1H, d).

### b) 7-Hydroxy-8-nitro-1,2,4,5-tetrahydro-benzo[d]azepine-3-carboxylic acid tert-butyl ester (D16b)

Nitration of D16a was carried out by adding 70% aqueous nitric acid (8 g) dissolved in glacial acetic acid (100 ml)/acetic anhydride (10 ml) to the phenol D15a (20 g) dissolved in AcOH (200 ml)/acetic anhydride (20 ml) at 0° C. Aqueous work-up followed by chromatography on silica gel using 0-20% EtOAc/n-hexane as eluant afforded the title compound D16b (11 g). ¹H NMR (CDCl₃) δ 7.85 (1H, s), 6.93 (1H, s), 3.56 (4H, m), 2.91 (4H, m), 1.48 (9H, m).

### c) 7-Nitro-8-trifluoromethanesulfonvloxy-1,2,4,5-tetrahydro-benzo[d]azepine-3-carboxylic acid tert-butyl ester (D16c)

D16b (8.4 g) was dissolved in acetone (300 ml) and cooled to 0° C. Trifluoromethanesulfonyl chloride (4.4 ml) was added and the resultant mixture stirred at room temperature for 2 h. Evaporation in vacuo followed by basic aqueous work-up afforded the title compound D16c (12 g). ¹H NMR (CDCl₃) δ 7.95 (1H, s), 7.19 (1H, s), 3.61 (4H, m), 3.02 (4H, m), 1.48 (9H, m).

### d) 7-Nitro-8-dimethylamino-1,2,4,5-tetrahydro-benzo[d]azepine-3-carboxylic acid tert-butyl ester (D16d)

A suspension of BINAP (106 mg), palladium acetate (26 mg) and caesium carbonate (556 mg) in dioxan (5 ml) under argon was sonicated for 30 min at room temperature. To the resulting red suspension was added D16c (500 mg) and dimethylamine hydrochloride (150 mg). The mixture was then heated in a microwave reactor for 30 mins at 160° C., diluted with diethyl ether (30 ml) and washed with water (50 ml) and saturated sodium bicarbonate solution (30 ml) and then the layers separated. The organic portion was dried (Na₂SO₄), filtered and evaporated to give the title compound D16d as an oil (263 mg). MH⁺ 336.

### e) 7-Amino-8-dimethylamino-1,2,4,5-tetrahydro-benzo[d]azepine-3-carboxylic acid tert-butyl ester (D16)

Hydrogenation of D19a at 50 psi in ethanol over 10% palladium on charcoal at room temperature afforded the title compound D16. MH⁺ 306

Description 17

### 7-Methoxy-8-(4-nitro-benzenesulfonylamino)-1,2,4,5-tetrahydro-3-benzazepine-3-carboxylic acid tert-butyl ester (D17)

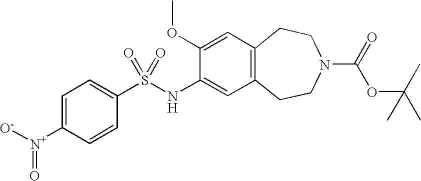

7-amino-8-methoxy-1,2,4,5-tetrahydro-3-benzazepine-3-carboxylic acid tert-butyl ester (3 g, 10.3 mmol) was dissolved in dry pyridine (15 ml) and the solution was cooled to 0° C. 4-nitro-benzenesulfonyl chloride, previously dissolved in the required amount of dry dichloromethane, (1.1 eq., 11.3 mmol, 2.5 g) was added to the pyridine solution at 0° C. The reaction mixture was stirred at room temperature for 6 hours. The mixture was then diluted with dichloromethane (100 ml) and poured onto water (100 ml); the organics were separated from the aqueous and they were washed with citric acid aqueous solution (10%) (100 ml×2), then with brine (100 ml); the organics were then dried over Na₂SO₄, filtered and the organic solvent was evaporated to afford the crude product.

Chromatography on silica eluting with 0-5% MeOH-dichloromethane afforded the title compound as a yellow solid, 4.13 g, 84%. M⁺−1H=475.

Description 18

### N-(8-Methoxy-2,3,4,5-tetrahydro-1H-3-benzazepin-7-yl)-4-nitro-benzenesulfonamide (D18)

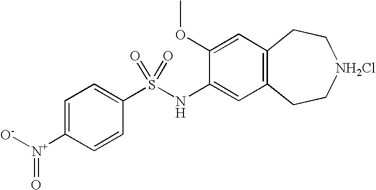

7-Methoxy-8-(4-nitro-benzenesulfonylamino)-1,2,4,5-tetrahydro-3-benzazepine-3-carboxylic acid tert-butyl ester (4.13 g, 8.66 mmol) was dissolved in EtOAc (100 ml) and HCl (4M solution in 1,4-dioxane) (20 ml) was added at room temperature; the reaction mixture was stirred overnight at room temperature; the solvent was then evaporated to give the crude product, 3.5 g, which was directly used for the next step. M⁺=377

Description 19

### N-(8-Methoxy-3-methyl-2,3,4,5-tetrahydro-1H-3-benzazepin-7-yl)-4-nitro-benzenesulfonamide (D19)

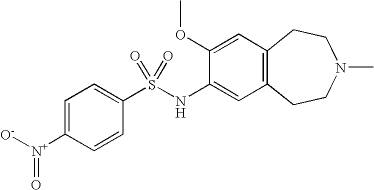

N-(8-Methoxy-2,3,4,5-tetrahydro-1H-3-benzazepin-7-yl)-4-nitro-benzenesulfonamide D18 (3.5 g, 8.6 mmol), was dissolved in 1,2-dichloroethane (200 ml) and Et₃N (2 eq., 17.3 mmol, 1.75 g, 2.4 ml), CH₂O (37% aqueous solution) (7.6 eq., 65.8 mmol, 1.98 g, 5.3 ml), were added at room temperature and the mixture was stirred at room temperature for 30 minutes; NaBH(OAc)₃ (3.2 eq., 27.7 mmol, 5.9 g) was subsequently added at room temperature to the reaction mixture that was all stirred at room temperature for 2 hours. The mixture was poured onto NaHCO₃ (sat. solution) (100 ml) very slowly. The two phases were separated and the organics were washed with NaHCO₃ (sat. solution) (100 ml) and brine (100 ml); the organics were then dried over Na₂SO₄, filtered and the organic solvent was evaporated to afford the crude product. Dichloromethane (20 ml) was added to the crude product, and yellow solid crystals developed from the solution; the crystals were filtered and dried to afford the title compound as a yellow solid, 3.3 g, 97%. M⁺=391.

Description 20

### 4-Amino-N-(8-methoxy-3-methyl-2,3,4,5-tetrahydro-1H-3-benzazepin-7-yl)-benzenesulfonamide (D20)

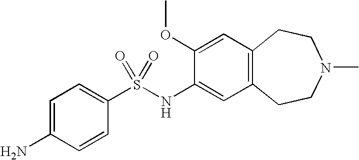

N-(8-Methoxy-3-methyl-2,3,4,5-tetrahydro-1H-3-benzazepin-7-yl)-nitro-benzene-sulfonamide D19 (3.3 g, 8.4 mmol) was dissolved in EtOH (100 ml) and Pd/C (10%, 0.33 g), was added; the reaction mixture was hydrogenated at room temperature at 50 psi for 18 h; the reaction mixture was subsequently filtered through celite and the solvent was evaporated to afford the title compound as a pale yellow solid, 36%. M⁺=361

Description 21

### 7-(4-Bromomethyl-benzenesulfonylamino)-8-methoxy-1,2,4,5-tetrahydro-3-benzazepine-3-carboxylic acid tert-butyl ester (D21)

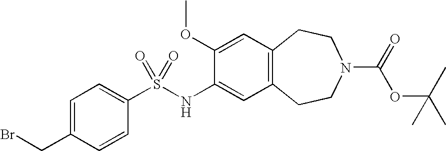

7-amino-8-methoxy-1,2,4,5-tetrahydro-3-benzazepine-3-carboxylic acid tert-butyl ester D5 (0.1 g, 0.342 mmol) was dissolved in dry dichloromethane (5 ml) and dry pyridine (1 eq., 0.342 mmol, 0.027 ml) was added; the solution was cooled to 0° C. and 4-Bromomethyl-benzenesulfonyl chloride, previously dissolved in the required amount of dry dichloromethane, (1.1 eq., 0.377 mmol, 0.102 g) was added to the solution at 0° C. The reaction mixture was stirred at room temperature overnight. The mixture was then diluted with dichloromethane (10 ml) and poured onto water (100 ml); the organics were separated from the aqueous and they were washed with citric acid aqueous solution (10%) (20 ml×2), then with brine (20 ml); the organics were subsequently dried over Na₂SO₄, filtered and the organic solvent was evaporated to afford the crude product. Chromatography on silica eluting with 0-30% hexane-ethyl acetate afforded a mixture of the title compound and its chloro analogue, 7-(4Chloromethyl-benzenesulfonylamino)-8-methoxy-1,2,4,5-tetrahydro-3-benzazepine-3-carboxylic acid tert-butyl ester. This mixture (0.116 9, 65%) was directly used for the next step. M⁺−H=524.

Description 22

### 7-{4-[(4-Fluoro-phenylamino)-methyl]-benzenesulfonylamino}-8-methoxy-1,2,4,5-tetrahydro-3-benzazepine-3-carboxylic acid tert-butyl ester (D22)

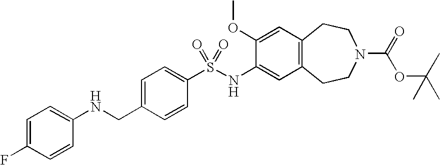

7-(4-Bromomethyl-benzenesulfonylamino)-8-methoxy-1,2,4,5-tetrahydro-3-benzazepine-3-carboxylic acid tert-butyl ester D21 (1 g, 1.9 mmol) was dissolved in CH₃CN (40 ml) and 4-fluoro-phenylamine (1.3 eq., 2.48 mmol, 0.274 g) followed by NaHCO₃ (4.35 eq., 8.27 mmol, 0.694 g) were added at room temperature; the reaction mixture was heated to 90° C. for 48 hours; the reaction mixture was cooled to room temperature and it was poured onto water (100 ml); the aqueous solution was extracted with EtOAc (100 ml×3), the organics were washed with brine (100 ml), dried over Na₂SO₄, filtered and the solvent was evaporated to afford the crude product. Chromatography on silica eluting with 0-90% hexane-ethyl acetate afforded the title compound as a white solid, 506 mg, 48%. M⁺−H=554

Description 23

### 7-(4-Carboxy-benzenesulfonylamino)-1,2,4,5-tetrahydro-3-benzazepine-3-carboxylic acid tert-butyl ester (D23)

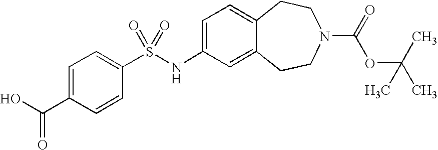

A stirred solution of 7-amino-1,2,4,5-tetrahydro-3-benzazepine-3-carboxylic acid tert-butyl ester D5 (3.0 g, 0.0114 mol) and pyridine (2 ml, 0.025 mol) in dry dicloromethane (50 ml) was treated with 4-chlorosulphonylbenzoic acid (3.03 g, 0.0137 mol) portionwise. The mixture was stirred at room temperature for 4 hours, then it was evaporated to dryness in vacuo, azeotroping with toluene, to give the title compound in crude form as a pale pink solid (5.6 g, >100%). MS (ES): m/z=445, MH⁺.

Description 24

### 7-(4-Hydroxymethyl-benzenesulfonylamino)-1,2,4,5-tetrahydro-3-benzazepine-3-carboxylic acid tert-butyl ester (D24)

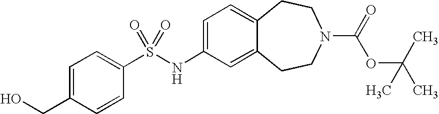

A suspension of 7-(4-carboxy-benzenesulfonylamino)-1,2,4,5-tetrahydro-3-benzazepine-3-carboxylic acid tert-butyl ester D23 (0.0114 mol) in dry tetrahydrofuran (75 ml) was stirred under an atmosphere of argon and cooled in an ice bath. Borane-tetrahydrofuran complex (1.0 M in tetrahydrofuran, 34.2 ml, 0.0342 mol) was added portionwise over 1 hour. The mixture was stirred at room temperature for 5 days, then it was quenched by the cautious addition of saturated ammonium chloride solution (50 ml) and extracted with ethyl acetate (100 ml, 2×50 ml). The combined extracts were dried (Na₂SO₄) and concentrated in vacuo to give a white foam (6.3 g) which was purified by silica chromatography eluting with dichloromethane then 1% then 2% then 5% methanol in dichloromethane. The product containing fractions were combined and evaporated to dryness to give the title compound as a white solid (4.74 g, 96%). MS (ES): m/z=431, M−H.

Description 25

### 7-(4-Methanesulfonyloxymethyl-benzenesulfonylamino)-1,2,4,5-tetrahydro-3-benzazepine-3-carboxylic acid tert-butyl ester (D25)

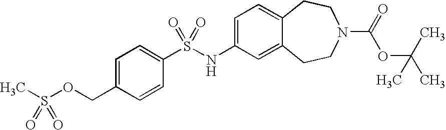

A stirred solution of 7-(4-hydroxymethyl-benzenesulfonylamino)-1,2,4,5-tetrahydro-3-benzazepine-3-carboxylic acid tert-butyl ester D24 (888 mg, 2.05 mmol) and triethylamine (0.3 ml, 2.26 mmol) in dry dichloromethane (10 ml) was cooled in an ice bath and treated dropwise with methanesulfonyl chloride (0.165 ml, 2.13 mmol). The mixture was stirred for 3 hours, then it was washed with water (10 ml) and the aqueous was back-extracted with dichloromethane (2×10 ml). The combined organics were dried (Na₂SO₄) and concentrated in vacuo to give a beige foam which was purified by silica chromatography eluting with 0.5% then 2% methanol in dichloromethane. The product-containing fractions were combined and evaporated to dryness to give the title compound as a white foam (688 mg, 66%). MS (ES): m/z=509, M−1.

Description 26

### b 7-[4-(4-Chloro-phenoxymethyl)-benzenesulfonylamino-1,2,4,5-tetrahydro-3-benzazepine-3-carboxylic acid tert-butyl ester (D26)

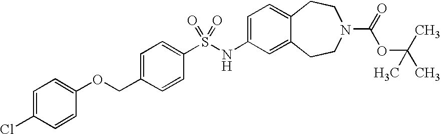

A solution of 4-chlorophenol (151 mg, 1.18 mmol) in N,N-dimethylformamide (2 ml) was treated with sodium hydride (60% in oil, 47 mg, 1.18 mmol) and the mixture was stirred at room temperature for 30 minutes. A solution of 7-(4-methanesulfonyloxymethyl-benzenesulfonylamino)-1,2,4,5-tetrahydro-3-benzazepine-3-carboxylic acid tert-butyl ester D25 (300 mg, 0.59 mmol) in N,N-dimethylformamide (2 ml) was added and the mixture was stirred at room temperature for 2 hours. Saturated ammonium chloride solution (5 ml aqueous) was added and the mixture was extracted with ethyl acetate (3×10 ml). The combined organics were dried (Na₂SO₄) and concentrated in vacuo to give a residue which was purified by silica chromatography eluting with 0.5% then 1% methanol in dichloromethane. The product-containing fractions were combined and evaporated to dryness to give the title compound as a colourless glass (94 mg, 30%). MS (ES): m/z=543/545, MH⁺.

Description 27

### 7-(4-Carboxy-benzenesulfonylamino)-8-dimethylamino-1,2,4,5-tetrahydro-3-benzazepine-3-carboxylic acid tert-butyl ester (D27)

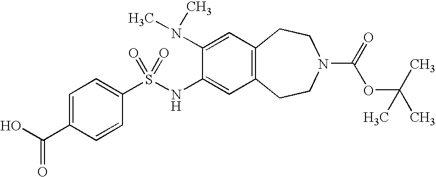

A solution of 8-amino-7-dimethylamino-1,2,4,5-tetrahydro-3-benzazepine-3-carboxylic acid tert-butyl ester D16 (1.0 g, 3.27 mmol) and pyridine (0.53 ml, 6.55 mmol) in dry dichloromethane (10 ml) was treated with 4-chlorosulfonylbenzoic acid (800 mg, 3.63 mmol) and the mixture was stirred at room temperature overnight. The solvent was removed by evaporation in vacuo, azeotroping with toluene, to give a residue which was purified by silica chromatography eluting with 1% then 2% then 5% then 10% then 20% methanol in dichloromethane. The product-containing fractions were combined and evaporated to dryness to give the title compound as a foam (1.24 g, 77%). MS (ES): m/z=490, MH⁺.

Description 28

### 7-{4-[(4-Chloro-phenyl)-methyl-carbamoyl]-benzenesulfonylamino)-8-dimethylamino-1,2,4,5-tetrahydro-3-benzazepine-3-carboxylic acid tert-butyl ester (D28)

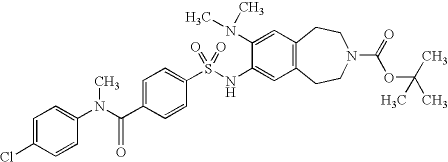

A mixture of 7-(4-carboxy-benzenesulfonylamino)-8-dimethylamino1,2,4,5-tetrahydro-3-benzazepine-3-carboxylic acid tert-butyl ester D27 (150 mg, 0.306 mmol), 4-chloro-N-methylaniline (173 mg, 1.22 mmol), 1-ethyl-3-(3-dimethylaminopropyl)carbodiimide hydrochloride (240 mg, 1.25 mmol), 1-hydroxybenzotriazole (172 mg, 1.27 mmol) and triethylamine (0.045 ml, 0.323 mmol) in N,N-dimethylformamide (3 ml) was stirred at room temperature overnight. Half-saturated ammonium chloride solution (10 ml) was added and the mixture was extracted with diethyl ether (3×10 ml). The combined organics were concentrated in vacuo to give a residue which was purified by silica chromatography eluting with dichloromethane followed by 1% then 2% methanol in dichloromethane. The product-containing fractions were combined and evaporated to dryness to give the title compound as a glass (119 mg, 63%). MS (ES): m/z=613/615, MH⁺.

### EXAMPLE 1

### 4-Benzyloxy-N-(8-bromo-2,3,4,5-tetrahydro-1H-benzo[d]azepin-7-yl)-benzenesulfonamide (E1)

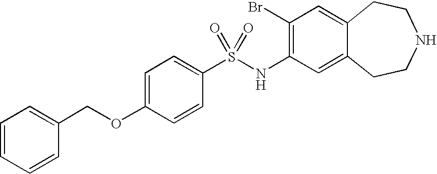

### a) 7-(4-Benzyloxy-benzenesulfonylamino)-8-bromo-1,2,4,5-tetrahydro-benzo[d]azepine-3-carboxylic acid tert butyl ester

A solution of 4-Benzyloxy-benzenesulfonyl chloride (D10) (190 mg, 0.6 mmol) in dichloromethane (5 mL) was added dropwise to a solution of D2 (150 mg, 0.44 mmol) in pyridine (5 mL) at 0° C. The mixture was stirred at room temperature for 18 h, then poured onto brine and extracted with ethyl acetate (×2). The combined organic layer was washed with citric acid, sodium bicarbonate solution and brine, then dried and evaporated to afford the crude product. Chromatography on silica, eluting with 23% ethyl acetate/hexane afforded the product (300 mg). MH⁺ 621.

### b) 4-Benzyloxy-N-(8-bromo-2,3,4,5-tetrahydro-1H-benzo[d]azepin-7-yl)-benzenesulfonamide hydrochloride

The title compound was prepared from a) by treatment with a solution of hydrogen chloride in dioxan (4M), followed by the addition of ether to precipitate the product. MH⁺487. ¹H NMR: δ DMSO 2.9-3.0 (4H, m), 3.12 (4H, m), 5.19 (2H, s), 7.06 (1H, s), 7.14 (2H, d), 7.43 (1H, s), 7.48 (4H, s), 7.63 (2H, d), 9.12 (2H, b s) 9.70 (1H, b s).

### EXAMPLE 2

### 4-Benzyloxy-N-(8-bromo-3-methyl-2,3,4,5-tetrahydro-1H-benzo[d]azepin-7-yl)-benzenesulfonamide hydrochloride (E2)

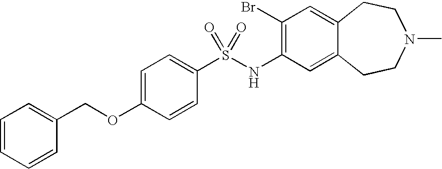

A solution of E1 (170 mg, 0.3 mmol) in dichloroethane (10 mL) containing triethylamine (0.5 mL) was treated with formalin (0.5 mL) followed by sodium triacetoxyborohydride (300 mg). The mixture was stirred for 1 h, then added to sodium bicarbonate solution and extracted with dichloromethane. The combined organic extracts were washed with brine, dried and evaporated to afford the crude product. Chromatography on silica, eluting with 2% methanol in dichloromethane containing 0.5% aqueous ammonia, afforded the title compound (140 mg) which was converted to the hydrochloride salt was by treatment with ethereal hydrogen chloride. MH⁺ 535. ¹H NMR: δ DMSO 2.77 (3H, s), 2.95 (4H, m), 3.23 (2H, m), 3.51 (2H, m), 5.18 (2H, s), 7.08 (1H, s), 7.14 (2H, d), 7.45 (1H, s), 7.48 (4H, s), 7.63 (2H, d), 9.70 (1H, b s), 11.00 (1H, b s).

Examples 3-70 were prepared using analogous procedures to Examples 1 and 2 using the anilines D1-D9, D15 or D16 and the appropriate sulfonyl chloride D11, D12, D13 or D14. Products were isolated as either the free bases or hydrochloride salts. All ¹H NMR are consistent with the structures shown.

### EXAMPLE 71

### 4-(4-Chloro-phenoxymethyl)-N-(3-methyl-2,3,4,5-tetrahydro-1H-3-benzazepin-7-yl)-benzenesulfonamide hydrochloride (E71)

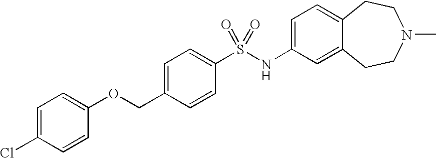

A solution of 7-[4-(4-chloro-phenoxymethyl)-benzenesulfonylamino]-1,2,4,5-tetrahydro-3-benzazepine-3-carboxylic acid tert-butyl ester D26 (94 mg, 0.173 mmol) in absolute ethanol (1 ml) was treated with ethanolic hydrogen chloride solution (ca 9M solution, 1 ml) and the mixture was stirred at room temperature for 5 hours. The solvent was removed by evaporation in vacuo and the residue was basified with saturated sodium bicarbonate solution (10 ml) and extracted with dichloromethane. Some insoluble matter was collected by filtration and this was combined with the material obtained from evaporation of the organic extracts. A suspension of this material in 1,2-dichloroethane (3 ml) was treated with formaldehyde (37% aqueous solution, 0.03 ml) and stirred for 30 minutes. Sodium triacetoxyborohydride (80 mg, 0.37 mmol) was added and the mixture was stirred overnight. More formaldehyde (0.1 ml) was added and after stirring for 30 minutes, more sodium triacetoxyborohydride (250 mg) was added and the mixture was stirred for another 2 hours. Saturated sodium bicarbonate solution (10 ml) was added and the mixture was extracted with dichloromethane. The combined organics were concentrated in vacuo to give a residue which was purified by silica chromatography eluting with a gradient of 2% then 5% then 10% methanol in dichloromethane. The product-containing fractions were combined and evaporated to dryness to give the title compound as a colourless glass (47 mg, 59%). Converted to the hydrochloride salt by treatment with I.OM ethereal hydrogen chloride solution. MS (ES): m/z=457/459, MH⁺.

Examples 72-82 were prepared using analogous procedures to Example 71 and Descriptions 23-26 using the appropriately 8-substituted benzazepine. Products were isolated as either the free bases or hydrochloride salts. All ¹H NMR are consistent with the structures shown.

### EXAMPLE 83

### 4-(4-Fluoro-benzylamino)-N-(8-methoxy-3-methyl-2,3,4,5-tetrahydro-1H-3-benzazepin-7-yl)-benzenesulfonamide hydrochloride (E83)

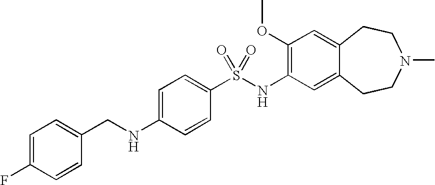

4-Amino-N-(8-methoxy-3-methyl-2,3,4,5-tetrahydro-1H-3-benzazepin-7-yl)-benzene-sulfonamide D20 (0.1 g, 0.277 mmol) was dissolved in 1,2-dichloroethane (10 ml) and 4-fluoro-benzaldehyde (6 eq., 1.66 mmol, 0.206 g, 0.178 ml) was added; NaBH(OAc)₃ (3.2 eq., 0.886 mmol, 0.187 g) was added at room temperature and the mixture was stirred at room temperature for 48 hours. The mixture was poured onto NaHCO₃ (sat. solution) (10 ml) and stirred for 10 minutes; the two phases were separated and the organics were then dried over Na₂SO₄, filtered and the solvent was evaporated to afford the crude product; Chromatography on silica eluting with 0-10% MeOH/NH₃-dichloromethane afforded the title compound as a white solid, 30 mg, 23%, which was converted to the hydrochloride salt. MH⁺=470.

Examples 84-87 were prepared using analogous procedures to Example 83 using the anilines D20 and the appropriate benzaldehyde. Products were isolated as either the free bases or hydrochloride salts. All ¹H NMR are consistent with the structures shown.

### EXAMPLE 88

### [(4-Fluoro-benzyl)-methyl-amino]-N-(8-methoxy-3-methyl-2,3,4,5-tetrahydro-1H-3-benzazepin-7-yl)-benzenesulfonamide hydrochloride (E88)

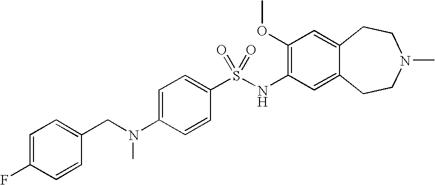

### a) [(4-Fluoro-benzyl)-formyl-amino]-N-(methoxy-methyl-2,3,4,5-tetrahydro-1H-3-benzazepin-7-yl)-benzenesulfonamide

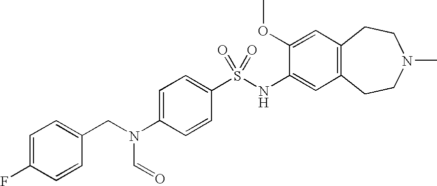

4-(4-Fluoro-benzylamino)-N-(8-methoxy-3-methyl-2,3,4,5-tetrahydro-1H-3-benzazepin-7-yl)-benzenesulfonamide E82 (660 mg, 1.45 mmol) was dissolved in formic acid (50 ml) and the reaction mixture was refluxed at 70° C. overnight. The reaction mixture was then cooled to room temperature and the solvent was evaporated to afford the crude title compound which was directly used for the next step. MH⁺=498

### b) [(4-Fluoro-benzyl)-methyl-amino]-N-(methoxy-methyl-2,3,4,5-tetrahydro-1H-3-benzazepin-7-yl)-benzenesulfonamide-7-yl)-benzenesulfonamide hydrochloride

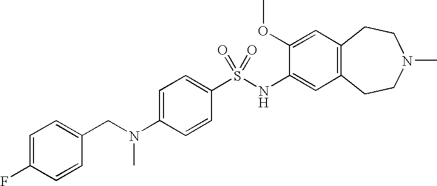

[(4-Fluoro-benzyl)-formyl-amino]-N-(methoxy-methyl-2,3,4,5-tetrahydro-1H-3-benzazepin-7-yl)-benzenesulfonamide from part a) (0.128 g, 0.260 mmol) was dissolved in dry THF (7 ml) and BF₃ OEt₂ (5.2 eq., 1.35 mmol, 0.192 g, 0.166 ml) was added at room temperature; BH₃-THF (1M solution) (7.5 eq., 1.95 mmol, 1.95 ml) was then added at room temperature and the reaction mixture was stirred at 70° C. for 48 hours; the reaction was cooled to room temperature and stirred at room temperature overnight; MeOH (13 ml) was added and the mixture was refluxed for 2 further hours. When cooled to room temperature, the crude mixture was purified by SCX to afford the title compound as a white solid, 62 mg, 50 %, and converted to the hydrochloride salt. MH⁺=484

### EXAMPLE 89

### 4-[(4Fluoro-phenylamino)-methyl]-N-(8-methoxy-3-methyl-2,3,4,5-tetrahydro-1H-3-benzazepin-7-yl)-benzenesulfonamide hydrochloride (E89)

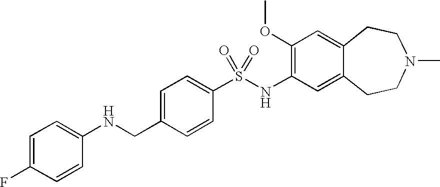

### a) 4-[(4-Fluoro-phenylamino)-methyl]-N-(8-methoxy-2,3,4,5-tetrahydro-1H-3-benzazepin-7-yl)-benzenesulfonamide hydrochloride

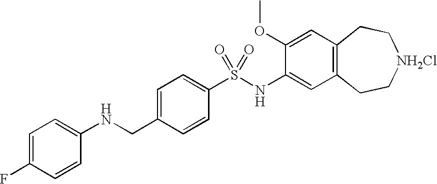

7-{4-[(4-Fluoro-phenylamino)-methyl]-benzenesulfonylamino}-8-methoxy-1,2,4,5-tetrahydro-3-benzazepine-3-carboxylic acid tert-butyl ester D22 (0.506 g, 0.911 mmol) was dissolved in EtOAc (5 ml) and HCl (4M solution in 1,4-dioxane) (5 ml) was added at room temperature; the reaction mixture was stirred overnight at room temperature; the solvent was then evaporated to give the crude HCl salt, 0.440 g (reaction complete), which was used directly for the next step. MH⁺=456.

### b) 4-[(4-Fluoro-phenylamino)-methyl]-N-(8-methoxy-3-methyl-2,3,4,5-tetrahydro-1H-3-benzazepin-7-yl)-benzenesulfonamide hydrochloride

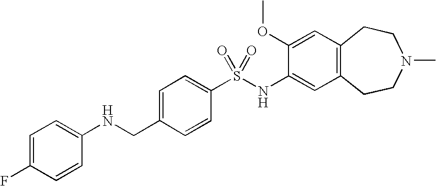

4-[(4-Fluoro-phenylamino)-methyl]-N-(8-methoxy-2,3,4,5-tetrahydro-1H-3-benzazepin-7-yl)-benzenesulfonamide) from part a) (0.440 g 0.911 mmol) was dissolved in 1,2-dichloroethane (10 ml) and Et₃N (1.5 eq., 1.36 mmol, 0.138 g, 0.190 ml), CH₂O (37% aqueous solution) (1.1 eq., 1 mmol, 0.03 g, 0.081 ml), were added at room temperature and the mixture was stirred at room temperature for 30 minutes; NaBH(OAc)₃ (1.5 eq., 1.37 mmol, 0.289 g) was subsequently added at room temperature to the reaction mixture and stirred at room temperature for 5 hours. The mixture was poured onto NaHCO₃ (sat. solution) (10 ml). The two phases were separated and the organics were dried over Na₂SO₄, filtered and the organic solvent was evaporated to afford the crude product.

Chromatography on silica eluting with 2-10% MeOH-dichloromethane afforded the title compound, which was converted to the hydrochloride salt. 100 mg, 23%. MH⁺=470.

Examples 90-91 were prepared using analogous procedures to Examples 89 and Description 22, using the appropriate aniline. Products were isolated as either the free bases or hydrochloride salts. All ¹H NMR are consistent with the structures shown.

### EXAMPLE 92

### 4{[(4-Fluoro-phenyl)-methyl-amino]-methyl)-N-(8-methoxy-3-methyl-2,3,4,5-tetrahydro-1H-3-benzazepin-7-yl)-benzenesulfonamide hydrochloride (E92)

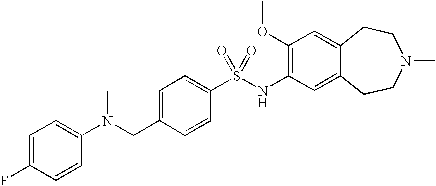

4-[(4-Fluoro-phenylamino)-methyl]-N-(8-methoxy-2,3,4,5-tetrahydro-1H-3-benzazepin-7-yl)-benzenesulfonamide) E90 (0.440 g 0.911 mmol) was dissolved in 1,2-dichloroethane (10 ml) and Et₃N (1.5 eq., 1.36 mmol, 0.138 g, 0.190 ml), CH₂O (37% aqueous solution) (1.1 eq., 1 mmol, 0.03 g, 0.081 ml), were added at room temperature: the mixture was stirred at room temperature for 30 minutes; NaBH(OAc)₃ (1.5 eq., 1.37 mmol, 0.289 g) was subsequently added at room temperature to the reaction mixture and stirred at room temperature for 5 hours. The mixture was poured onto NaHCO₃ (sat. solution) (10 ml). The two phases were separated and the organics were dried over Na₂SO₄, filtered and the organic solvent was evaporated to afford the crude product. Chromatography on silica eluting with 2-10% MeOH-dichloromethane afforded the title compound, which was converted to the hydrochloride salt, 154 mg, 36%. MH⁺=484.

Examples 93-94 were prepared using an analogous procedure to Example 92. Products were isolated as either the free bases or hydrochloride salts. All ¹H NMR are consistent with the structures shown.

### EXAMPLE 95

### N-(8-Dimethylamino-3-methyl-2,3,4,5-tetrahydro-1H-3-benzazepin-7-yl)-4-(4-fluoro-benzyloxy)-benzenesulfonamide hydrochloride (E95)

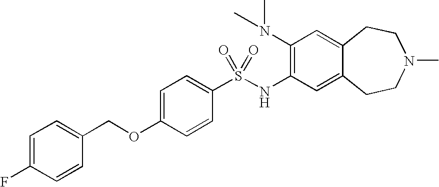

A solution of 8-amino-7-dimethylamino-1,2,4,5-tetrahydro-3-benzazepine-3-carboxylic acid tert-butyl ester D16 (100 mg, 0.327 mmol) and pyridine (0.06 ml, 0.74 mmol) in dry dichloromethane (2 ml) was treated with 4-(4-fluoro-benzyloxy)-benzenesulfonyl chloride D12 (120 mg, 0.40 mmol), stirred overnight at room temperature and then evaporated to dryness in vacuo. A suspension of the residue in absolute ethanol (2 ml) was treated with hydrogen chloride solution (4.0M in dioxan, 2 ml) and the mixture was stirred at room temperature for 5 hours, then heated to 40° C. for 4 hours. The solvent was removed in vacuo and the residue was suspended in 1,2 dichloroethane (2 ml) and treated with formaldehyde (37% aqueous solution, 0.5 ml). The mixture was stirred at room temperature for 15 minutes, then sodium triacetoxyborohydride (140 mg, 0.66 mmol) was added and the mixture was stirred for 2 days. Saturated sodium bicarbonate solution (10 ml) was added and the mixture was extracted with dichloromethane. The combined organics were concentrated in vacuo and the residue was purified by silica chromatography eluting with dichloromethane then 2% then 5% methanol in dichloromethane. The product-containing fractions were combined to give the title compound as a colourless residue (151 mg, 95%). Converted to the hydrochloride salt using 1.0M ethereal hydrogen chloride solution.

¹H NMR (CDCl₃, selected data for free base) δ: 2.36 (3H, s), 2.39 (6H, s), 2.45-2.6 (4H, m), 2.75-2.9 (4H, m), 5.02 (2H, s), 6.81 (1H, s), 6.95 (2H, d), 7.08 (2H, t), 7.30 (1H, s), 7.34 (2H, m), 7.76 (2H, d). MS (ES): m/z=484, MH⁺.

Examples 96-97 were prepared using an analogous procedure to Example 95 using the appropriate sulfonyl chloride. Products were isolated as either the free bases or hydrochloride salts. All ¹H NMR are consistent with the structures shown.

### EXAMPLE 98

### N-(8-Dimethylamino-3-methyl-2,3,4,5-tetrahydro-1H-3-benzazepin-7-yl)-[(4-fluoro-benzyl)-methyl-amino]-benzenesulfonamide hydrochloride (E98)

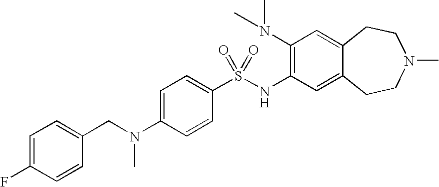

The title compound was prepared from 8-amino-7-dimethylamino-1,2,4,5-tetrahydro-3-benzazepine-3-carboxylic acid tert-butyl ester D16 by the method described for Example 88. MS (ES): m/z=497, MH⁺.

Example 99 was prepared using an analogous procedure to Example 98. Product was isolated as either the free base or hydrochloride salt. All ¹H NMR are consistent with the structures shown.

### EXAMPLE 100

### N-(Dimethylamino-methyl-2,3,4,5-tetrahydro-1H-3-benzazepin-7-yl)-4-{[(4-fluoro-phenyl)-methyl-amino]-methyl}-benzenesulfonamide hydrochloride (E100)

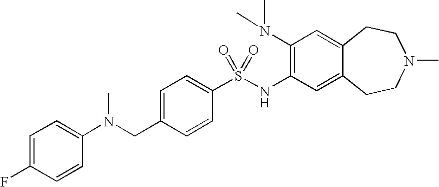

A solution of 7-{4-[(4-chloro-phenyl)-methyl-carbamoyl]-benzenesulfonylamino)-8-dimethylamino-1,2,4,5-tetrahydro-3-benzazepine-3-carboxylic acid tert-butyl ester (119 mg, 0.19 mmol) in dry tetrahydrofuran (2 ml) was stirred under argon and cooled in an ice bath. Lithium aluminum hydride (1.0M in tetrahydrofuran, 0.39 ml, 0.39 mmol) was added dropwise, and the mixture was stirred for 1 hour. Saturated ammonium chloride solution (5 ml) was added and the mixture was extracted with ethyl acetate (3×10 ml). The combined organics were dried (Na₂SO₄) and concentrated in vacuo to give a residue (130 mg) which was dissolved in absolute ethanol (2 ml) and treated with hydrogen chloride solution (4.0M in dioxan, 2 ml). This mixture was stirred at room temperature for 2.5 hours then the solvent was removed by evaporation in vacuo. The residue was partitioned between saturated sodium bicarbonate solution (10 ml) and dichloromethane. The organic phase was separated and applied to a pre-wetted SCX cartridge and eluted with methanol followed by ammonia/methanol. The ammonia/methanol fraction was concentrated in vacuo to give a residue (97 mg) which was taken up in 1,2-dichloroethane (2 ml) and then treated with formaldehyde solution (37% in water, 0.1 ml). The mixture was stirred at room temperature for 1 hour, then sodium triacetoxyborohydride (85 mg, 0.40 mmol) was added and the reaction was stirred for a further 2 hours. It was basified with saturated sodium bicarbonate solution (5 ml) and extracted with dichloromethane. The organics were concentrated in vacuo and purified by silica chromatography eluting with 2% then 5% methanol in dichloromethane. The product-containing fractions were combined and evaporated to dryness to give the title compound as a colourless gum (30 mg, 30%). Converted to the hydrochloride salt using 1.0M ethereal hydrogen chloride solution.

¹H NMR (CDCl₃, selected data for free base) δ: 2.35 (9H, m), 2.45-2.6 (4H, m), 2.75-2.9 (4H, m), 3.00 (3H, s), 4.50 (3H, s), 6.55 (2H, d), 6.80 (IH, s), 7.12 (2H, d), 7.25 (2H, d), 7.30 (1H, s), 7.77 (2H, d). MS (ES): m/z=513/515, MH⁺.

All the compounds listed below in Table 1 below relate to compounds of formulae (IA), (IB), (IC), (ID) and (IH):

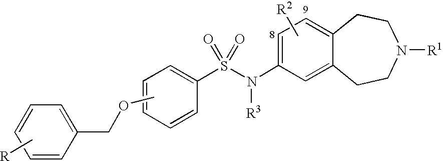

wherein m and n are both 2, Z is —CH₂O—, R⁴ is phenyl, R⁵ and R⁶ are hydrogen and R is a substituent on R⁴.

TABLE 1

| | | R² | R² | | Position | | |
|---|---|---|---|---|---|---|---|
| Example | R¹ | at C-9 | at C-8 | R³ | of ZR⁴ | R | MH⁺ |
| 1 | H | H | Br | H | para | H | 487 |
| 2 | Me | H | Br | H | para | H | 501 |
| 3 | H | H | H | H | para | H | 408 |
| 4 | Me | H | H | H | para | H | 422 |
| 5 | H | H | Et | H | para | H | 436 |
| 6 | Me | H | Et | H | para | H | 450 |
| 7 | H | H | MeO | H | para | H | 438 |
| 8 | Me | H | MeO | H | para | H | 452 |
| 9 | H | H | H | H | para | 4-Cl | 443 |
| 10 | Me | H | H | H | para | 4-Cl | 457 |
| 11 | H | H | Br | H | para | 4-Cl | 521 |
| 12 | Me | H | Br | H | para | 4-Cl | 535 |
| 13 | Me | H | Me | H | para | 4-Cl | 471 |
| 14 | Me | Cl | H | H | para | H | 457 |
| 15 | Me | Br | H | H | para | H | 501 |
| 16 | H | H | H | H | meta | H | 408 |
| 17 | Me | H | H | H | meta | H | 422 |
| 18 | H | H | Br | H | meta | H | 487 |
| 19 | Me | H | Br | H | meta | H | 501 |
| 20 | H | H | MeO | H | meta | H | 438 |
| 21 | Me | H | MeO | H | meta | H | 452 |
| 22 | H | H | Me | H | meta | H | 422 |
| 23 | Me | H | Me | H | meta | H | 436 |
| 24 | Me | Cl | H | H | meta | H | 457 |
| 25 | H | H | H | H | para | 3-Cl | 443 |
| 26 | Me | H | H | H | para | 3-Cl | 457 |
| 27 | H | H | H | H | para | 4-F | 426 |
| 28 | Me | H | H | H | para | 4-F | 440 |
| 29 | H | H | H | H | para | 2-Me | 422 |
| 30 | Me | H | H | H | para | 2-Me | 436 |
| 31 | H | H | H | H | para | 3-Me | 422 |
| 32 | Me | H | H | H | para | 3-Me | 436 |
| 33 | H | H | H | H | para | 3,4-diF | 444 |
| 34 | Me | H | H | H | para | 3,4-diF | 458 |
| 35 | H | H | H | H | para | 2,4-diF | 444 |
| 36 | Me | H | H | H | para | 2,4-diF | 458 |
| 37 | H | H | Br | H | para | 4-F | 505 |
| 38 | Me | H | Br | H | para | 4-F | 519 |
| 39 | H | H | OMe | H | para | 4-F | 456 |
| 40 | Me | H | OMe | H | para | 4-F | 470 |
| 41 | H | H | Cl | H | para | 4-F | 461 |
| 42 | Me | H | Cl | H | para | 4-F | 475 |
| 45 | Me | H | H | H | para | 2-F | 441 |
| 46 | Me | H | H | H | para | 3-F | 441 |
| 47 | Me | H | H | H | para | 4-CF₃ | 491 |
| 48 | Me | H | H | H | para | 4-Me | 437 |
| 49 | Me | H | Br | H | para | H | 502 |
| 50 | Me | H | Cl | H | para | H | 457 |
| 51 | Me | H | Me | H | para | H | 437 |
| 52 | Me | H | MeO | H | para | 3-F | 471 |
| 53 | Me | H | Cl | H | para | 4-Cl | 492 |
| 54 | Me | H | Br | H | para | 3-F | 520 |
| 55 | Me | H | Br | H | para | 2-F | 520 |
| 56 | Me | H | Br | H | para | 3,4-diF | 538 |
| 57 | Me | H | Br | H | para | 4-Me | 516 |
| 58 | Me | H | Br | H | para | 4-Br | 581 |
| 59 | Me | H | Br | H | para | 4-CF₃ | 570 |
| 60 | Me | H | H | H | meta | 4-F | 441 |
| 61 | Me | H | H | H | meta | 4-Cl | 457 |
| 62 | Me | H | MeO | H | meta | 4-F | 471 |
| 63 | Me | H | Br | H | meta | 4-Cl | 536 |
| 64 | Me | H | Br | H | meta | 4-F | 520 |
| 65 | Me | H | H | H | ortho | 4-F | 441 |
| 66 | Me | H | MeO | H | ortho | 4-F | 471 |
| 67 | Me | H | Br | H | ortho | 4-F | 520 |
| 68 | Me | H | H | H | ortho | 4-Cl | 457 |
| 69 | Me | H | MeO | H | ortho | 4-Cl | 487 |
| 70 | Me | H | i-Pr | H | ortho | H | 465 |

All of the compounds listed below in Table 2 below relate to compounds of formulae (IA), (IB), (IC), (ID) and (IF):

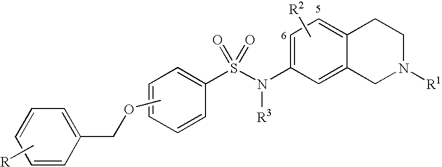

wherein m is 2 and n is 1, Z is —CH₂O—, R⁴ is phenyl, R⁵ and R⁶ are hydrogen and R is a substituent on R⁴.

TABLE 2

| | | R² | R² | | Position | | |
|---|---|---|---|---|---|---|---|
| Example | R¹ | at C-5 | at C-6 | R³ | of ZR⁴ | R | MH⁺ |
| 43 | H | H | H | H | para | H | 395 |
| 44 | H | H | MeO | H | para | H | 425 |

All of the compounds listed below in Table 3 below relate to compounds of formulae (IA), (IB), (IC) and (IH)

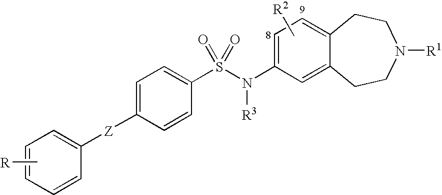

wherein m and n are both 2, R⁴ is phenyl, R⁵ and R⁶ are hydrogen and R is a substituent on R⁴.

TABLE 3

| | | R² | R² | | | | |
|---|---|---|---|---|---|---|---|
| Example | R¹ | at C-9 | at C-8 | R³ | Z | R | MH⁺ |
| 71 | Me | H | H | H | OCH₂ | 4-Cl | 457 |
| 72 | Me | H | H | H | OCH₂ | H | 423 |
| 73 | Me | H | H | H | OCH₂ | 4-F | 441 |
| 74 | Me | H | MeO | H | OCH₂ | H | 453 |
| 75 | Me | H | MeO | H | OCH₂ | 4-Cl | 487 |
| 76 | Me | H | MeO | H | OCH₂ | 4-F | 471 |
| 77 | Me | H | Br | H | OCH₂ | H | 502 |
| 78 | Me | H | Br | H | OCH₂ | 4-Cl | 536 |
| 79 | Me | H | Br | H | OCH₂ | 4-F | 520 |
| 80 | Me | H | i-PrO | H | OCH₂ | H | 481 |
| 81 | Me | H | i-PrO | H | OCH₂ | 4-Cl | 515 |
| 82 | Me | H | i-PrO | H | OCH₂ | 4-F | 499 |
| 83 | Me | H | MeO | H | CH₂NH | 4-F | 470 |
| 84 | Me | H | MeO | H | CH₂NH | 4-Cl | 486 |
| 85 | Me | H | MeO | H | CH₂NH | 4-MeO | 482 |
| 86 | Me | H | MeO | H | CH₂NH | 4-CN | 477 |
| 87 | Me | H | MeO | H | CH₂NH | 4-Ac | 459 |
| 88 | Me | H | MeO | H | CH₂NMe | 4-F | 484 |
| 89 | Me | H | MeO | H | NHCH₂ | 4-F | 470 |
| 90 | Me | H | MeO | H | NHCH₂ | 4-Cl | 486 |
| 91 | Me | H | MeO | H | NHCH₂ | 4-MeO | 482 |
| 92 | Me | H | MeO | H | NMeCH₂ | 4-F | 484 |
| 93 | Me | H | MeO | H | NMeCH₂ | 4-Cl | 500 |
| 94 | Me | H | MeO | H | NMeCH₂ | 4-MeO | 496 |
| 95 | Me | H | Me₂N | H | CH₂O | 4-F | 484 |
| 96 | Me | H | Me₂N | H | CH₂O | 3-F | 484 |
| 97 | Me | H | Me₂N | H | CH₂O | 4-CF₃ | 534 |
| 98 | Me | H | Me₂N | H | CH₂NMe | 4-F | 497 |
| 99 | Me | H | Me₂N | H | CH₂NMe | 4-Cl | 513 |
| 100 | Me | H | Me₂N | H | NMeCH₂ | 4-Cl | 513 |

All publications, including but not limited to patents and patent applications, cited in this specification are herein incorporated by reference as if each individual publication were specifically and individually indicated to be incorporated by reference herein as though fully set forth.

1-19. (canceled)

20. A compound of formula (I): 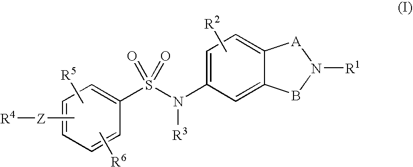
wherein
A and B represent the groups —(CH₂)ₘ— and —(CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or optionally substituted heterocyclyl;
R³ represents hydrogen or C₁₋₆alkyl;
R⁴ represents optionally substituted aryl or optionally substituted heteroaryl;
R⁵ and R⁶ each independently represent hydrogen, halogen, hydroxy, cyano, nitro, hydroxyC₁₋₆alkyl, trifluoromethyl, trifluoromethoxy, C₁₋₆alkyl, C₁₋₆alkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸, —(CH₂)ₚNR⁷COR⁸, optionally substituted aryl, optionally substituted heteroaryl or optionally substituted heterocyclyl;
R⁷ and R⁸ each independently represent hydrogen, C₁₋₆alkyl or, together with the nitrogen or other atoms to which they are attached, form an azacycloalkyl ring or an oxo-substituted azacycloalkyl ring;
m and n independently represent an integer selected from 1 and 2;
p independently represents an integer selected from 0, 1, 2 and 3;
and either:
Z represents —CR⁹R¹⁰X— or —XCR⁹R¹⁰— and X represents oxygen, sulfur, —SO— or —SO₂, or
Z represents —CONR¹¹— or —NR⁹CO—;
R⁹ and R¹⁰ each independently represent hydrogen, C₁₋₆alkyl or fluoro;
R¹¹ represents hydrogen or C₁₋₆alkyl;
or a pharmaceutically acceptable salt or solvate thereof.

21. A compound of formula (I) as claimed in claim 20 which is
4-benzyloxy-N-(8-bromo-2,3,4,5-tetrahydro-1H-benzo[d]azepin-7-yl)-benzenesulfonamide;
4-benzyloxy-N-(8-bromo-3-methyl-2,3,4,5-tetrahydro-1H-benzo [d]azepin-7-yl)-benzenesulfonamide;
4-(4-Chloro-phenoxymethyl)-N-(3-methyl-2,3,4,5-tetrahydro-1H-3-benzazepin-7-yl)-benzenesulfonamide;
4-(4-Fluoro-benzylamino)-N-(8-methoxy-3-methyl-2,3,4,5-tetrahydro-1H-3-benzazepin-7-yl)-benzenesulfonamide;
[(4-Fluoro-benzyl)-methyl-amino]-N-(8-methoxy-3-methyl-2,3,4,5-tetrahydro-1H-3-benzazepin-7-yl)-benzenesulfonamide;
4-[(4-Fluoro-phenylamino)-methyl]-N-(8-methoxy-3-methyl-2,3,4,5-tetrahydro-1H-3-benzazepin-7-yl)-benzenesulfonamide;
4-{[(4-Fluoro-phenyl)-methyl-amino]-methyl}-N-(8-methoxy-3-methyl-2,3,4,5-tetrahydro-1H-3-benzazepin-7-yl)-benzenesulfonamide;
4-{[(4-Fluoro-phenyl)-methyl-amino]-methyl}-N-(8-methoxy-3-methyl-2,3,4,5-tetrahydro-1H-3-benzazepin-7-yl)-benzenesulfonamide;
N-(8-Dimethylamino-3-methyl-2,3,4,5-tetrahydro-1H-3-benzazepin-7-yl)-4-(4-fluoro-benzyloxy)-benzenesulfonamide;
N-(8-Dimethylamino-3-methyl-2,3,4,5-tetrahydro-1H-3-benzazepin-7-yl)-[(4-fluoro-benzyl)-methyl-amino]-benzenesulfonamide;
N-(8-Dimethylamino-3-methyl-2,3,4,5-tetrahydro-1H-3-benzazepin-7-yl)-[(4-fluoro-benzyl)-methyl-amino]-benzenesulfonamide; and
N-(Dimethylamino-methyl-2,3,4,5-tetrahydro-1H-3-benzazepin-7-yl)-4-{[(4-fluoro-phenyl)-methyl-amino]-methyl}-benzenesulfonamide,
or pharmaceutically acceptable salts or solvates thereof.

22. A pharmaceutical composition comprising a compound of formula (I) as claimed in claim 20 or a pharmaceutically acceptable salt thereof and a pharmaceutically acceptable carrier therefor.

23. A method for the treatment of a condition which requires modulation of a dopamine receptor comprising administering to a subject in need thereof, a compound of formula (I) as claimed in claim 20, or a pharmaceutically acceptable salt or solvate thereof.

24. A method for the treatment of psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders comprising administering to a subject in need thereof, a compound of formula (I) as claimed in claim 20, or a pharmaceutically acceptable salt or solvate thereof.
